(19) United States
(12) Patent Application Publication
(10) Pub. No.: US 2024/0319210 A1
(43) Pub. Date: Sep. 26, 2024

(54) APPLICATION OF NEONATAL BILIARY ATRESIA BIOMARKER AND DETECTION METHOD FOR SAME

(71) Applicant: HUMAN METABOLOMICS INSTITUTE, INC., Shenzhen, Guangdong (CN)

(72) Inventors: Wei Jia, Shenzhen, Guangdong (CN); Guoxiang Xie, Shenzhen, Guangdong (CN); Kejun Zhou, Shenzhen, Guangdong (CN)

(21) Appl. No.: 18/267,469

(22) PCT Filed: Dec. 20, 2021
(86) PCT No.: PCT/CN2021/139607

§ 371 Date: Dec. 18, 2023

(30) Foreign Application Priority Data
Dec. 18, 2020 (CN) 202011506061.7
Dec. 18, 2020 (CN) 202011510885.1

(51) Int. Cl. G01N 33/72; G01N 30/02; G01N 30/72; G01N 30/88

(52) U.S. Cl. CPC G01N 33/728; G01N 30/7266; G01N 2030/027; G01N 2030/8822; G01N 2333/9108; G01N 2333/96419; G01N 2800/085

(57) ABSTRACT

Disclosed in the present invention is an application of conjugated bilirubin as a biomarker in preparation of a diagnosis device for evaluating a risk of neonatal biliary atresia. Also disclosed in the present invention are a bilirubin content measurement method, a stable quantitative detection reagent for bilirubin, and an application of the quantitative detection reagent for bilirubin in preparation of a kit for measuring a content of the bilirubin and evaluating a risk of biliary atresia of a subject, infant hepatitis syndrome, α1 antitrypsin deficiency disease or Alagille syndrome. Also disclosed in the present invention are a kit for measuring the content of the bilirubin or evaluating the risk of neonatal biliary atresia, an application of the kit, a method for evaluating the risk of neonatal biliary atresia, and a device for predicting the risk of neonatal biliary atresia by using an expression level of the conjugated bilirubin. The present invention has the advantages of being simple and rapid, flexible in sample preparation, low in limit of detection, good in repeatability and high in sensitivity; and the sample preparation and detection method is simple and easy to implement, low in cost and suitable for popularization and use.

The present application claims the priority of Chinese Patent Applications No. 2020115060617 and No. 2020115108851 filed on Dec. 18, 2020. The contents of the Chinese Patent Applications are incorporated herein by reference in their entireties.

### TECHNICAL FIELD

The present invention relates to the field of biomedical technology and analytical detection technology, particularly relates to biomarkers that may be used to assess the risk of neonatal biliary atresia and application in the field of monitoring, screening and diagnosis of neonatal biliary atresia thereof. The present invention also relates to methods and devices for detecting the biomarkers, particularly relates to qualitative and quantitative detection and analytical methods for the biomarkers.

### BACKGROUND

Biliary atresia (BA) is a progressive and idiopathic disease of the extrahepatic biliary duct system occurring in the neonatal period, and is a destructive inflammatory fibrous obstruction of the neonatal bile ducts. BA affects intrahepatic and extrahepatic bile ducts with various lengths, and is the leading cause of neonatal obstructive jaundice, as well as the leading cause of pediatric liver transplantation, which results in approximately 60% of pediatric liver transplantations. More than 80% of pediatric patients diagnosed with congenital biliary atresia will die from liver failure within 1 year of age if they are not treated promptly. Many pediatric patients with biliary atresia have severe liver fibrosis at the time of being diagnosed, which quickly progresses to cirrhosis and eventually results in liver failure. Therefore, early diagnosis of biliary atresia becomes increasingly urgent. Hepatoportoenterostomy (Kasai Procedure) is the first-choice treatment for biliary atresia. The timing of surgery has a significant impact on the efficacy of Kasai surgery. The studies have found that Kasai surgery performed within 30 or 45 days from birth significantly increases the rates of postoperative jaundice resolution and five-year autologous liver survival, and significantly reduces the demands for liver transplantation. However, unfortunately, biliary atresia currently cannot be diagnosed in a timely manner, and the surgery is usually performed after 2 months of birth.

Early screening and diagnosis of biliary atresia is of great significance to patients, which may save valuable treatment time and improve prognosis for the patients. Currently, common preoperative diagnostic methods in clinical practice include liver biopsy, cholangiopancreatography, hepatobiliography, duodenal duct examination, etc. However, these techniques have certain limitations. In imaging examination, abnormal gallbladder (shrunken gallbladder with poor contraction) and triangular cord sign are characteristic signs of the liver in pediatric patients suffering from biliary atresia by ultrasonography [Tan et al. Making the diagnosis of biliary atresia using the triangular cord sign and gallbladder length, Pediatr Radiol, 2000, 30(2):69-73]. Nevertheless, triangular cord sign may not be present in every pediatric patient, and the observed results of ultrasonography vary widely depending on different physicians, machines, etc. Therefore, the sensitivity and specificity reported for diagnosing biliary atresia vary. Recently, it is reported that the specificity of both gallbladder atresia and triangular cord sign by Meta-analysis may reach 99%, while the corresponding sensitivity is 28% and 80% [Liu et al, Value of γ-GT for early diagnosis of biliary atresia, National Medical Journal of China (Taipei), 1998, 61(12):716-720].

The high false-positive rate of biliary atresia diagnosed by magnetic resonance cholangiopancreatography is inevitable in infants less than 3 months of age due to the small bile duct diameter and little fluid in the bile duct. In addition, the pathological features of biliary atresia are small bile duct hyperplasia, bile embolism, cholestasis of capillary bile duct and hepatocyte, and periportal or perilobular fibrosis, and hepatic lobular structure is visible at early stage. Therefore, when it is clinically applied to distinguish biliary atresia from other neonatal cholestasis, it will result in delays in the diagnosis and treatment of biliary atresia due to the low accuracy of the detection or the complexity of the procedure. In the early stage of the disease, the clinical manifestations, biochemical indicators, imaging and histological features of BA overlap with other cholestatic diseases, and the diagnosis process is twisted, complicate, and costly, which easily leads to delayed diagnosis and increases the financial burden on the patient's family. Liver biopsy and duodenal duct examination will involve unnecessary surgery and cause additional harm to the suspected pediatric patients. Therefore, the search for simple and specific diagnostic methods never stops, and the search for simple and effective diagnostic biomarkers is of important significance for early screening and diagnosis of biliary atresia.

The present serological indicators for screening and diagnosis of biliary atresia include: 1) total serum bilirubin, direct bilirubin; used as an initial screening indicator. When neonatal serum total bilirubin >2.0 mg/dL (42-51 μmol/L) or direct bilirubin >1.0 mg/dL (17 μmol/L), screening for biliary atresia is required, but the specificity is low for the diagnosis of biliary atresia; 2) GGT: A large sample survey study shows that, in different age groups, the GGT levels are significantly higher in the biliary atresia group than those in other cholestatic disease groups [Chen X et al. Value of Gamma-Glutamyl Transpeptidase for Diagnosis of Biliary Atresia by Correlation With Age. J Pediatr Gastroenterol Nutr, 2016, 63(3):370-373]. Liu et al [Value of γ-GT for early diagnosis of biliary atresia, National Medical Journal of China (Taipei), 1998, 61(12):716-720] report that the accuracy the diagnosis of biliary atresia is 60%-85% when GGT >300 U/L. GGT is the most commonly used screening indicator for biliary atresia, but the accuracy is limited due to the large variation between pediatric patients in clinical cases. 3) Other indicators: serum bile acid, prothrombin concentration, and platelet determination are affected by many factors.

Biomarkers for the diagnosis of neonatal biliary atresia have been explored in the prior art. Chinese patent CN101221129B discloses a sulfonated bile acid enzyme fluorescence capillary analysis method and an enzyme fluorescence quantitative kit, which is suitable for rapid screening and diagnosing of hepatobiliary diseases, especially for early detection of neonatal jaundice and congenital biliary atresia. CN108267585A discloses MMP-7 in biological samples as a biomarker. CN102818866B discloses the ratio of taurine chenodeoxycholic acid and chenodeoxycholic acid in serum as a biomarker for neonatal biliary atresia.

Bilirubin is formed by the deironation of protohemes released after the destruction of erythrocytes. 80% of the bilirubin originates from the spleen and liver, while the remaining originates from the bone marrow. Bilirubin exists in three forms, indirect bilirubin or unconjugated bilirubin, conjugated bilirubin, and δ-bilirubin. Among them, conjugated bilirubin and δ-bilirubin may react directly with diazo reagents and are known as direct bilirubin. Conjugated bilirubin is produced by the combination of indirect bilirubin that enters the liver and the glucuronic acid under the action of the glucuronosyltransferase in liver. Bilirubin is an important basis for clinical determination of jaundice and an important indicator of liver function. Increased content of direct bilirubin is observed in various types of hepatitis, cirrhosis, and obstructive jaundice, and the bilirubin is more sensitive than transaminases. In severe anemia, the direct bilirubin content in serum is reduced. Therefore, the detection of bilirubin content has a very important clinical significance.

At present, the main biochemical methods for free bilirubin and conjugated bilirubin detection are: diazonium salt modified J-G method, bilirubin oxidase method, chemical oxidation method, vanadate method and transcutaneous bilirubin measurement method. Both the diazonium and oxidase methods are susceptible to the interferences from lipids and hemolysis. Meanwhile, traditional biochemical methods require a large volume of blood, and these methods have strict requirements for biological samples and can only detect conjugated bilirubin in serum or plasma. Transcutaneous bilirubin meters can only detect total bilirubin, but cannot distinguish free bilirubin and conjugated bilirubin.

Chinese patent application CN109991177A discloses a buffer reagent that can be used to determine the direct bilirubin content by oxidase method. Chinese patent application CN109541238A uses the reaction of oxidase and its substrate to generate hydrogen peroxide, then uses the reaction of hydrogen peroxide and peroxidase to release oxygen, which oxidizes the direct bilirubin in the solution into biliverdin, and further causes the absorption peak to drop at 450 nm. The degree of drop is recorded to calculate the direct bilirubin content. Chinese patent application CN108613976A discloses a detection kit for fully automatic biochemical analyzer. Chinese patent application CN111579703A establishes the qualitative and quantitative method for the three compounds of bilirubin oxidation products, BOX A, BOX B and hematinic acid, by liquid chromatography-tandem triple quadrupole mass spectrometer. The instrumental analysis method is optimized by the standards of the three compounds, including the optimization of liquid chromatography conditions, mass spectrometry detection conditions and mass spectrometry parameters.

The prior arts above provide quantitative determination of indirect bilirubin and direct bilirubin, but none of quantitative determination of each component of direct bilirubin is disclosed. The present invention aims to remedy the deficiencies of the prior arts by providing a method for the qualitative and quantitative analysis of the components in samples containing bilirubin. The method can be applied to assess the risk of neonatal biliary atresia conveniently and reliably, and it provides a feasible solution for rapid and efficient screening and diagnosis of clinical neonatal biliary atresia.

### SUMMARY

The present invention provides a technical solution that enables convenient, reliable, and efficient assessment, screening, and/or diagnosis of the risk of neonatal biliary atresia. The present invention provides a biomarker for assessing the risk of neonatal biliary atresia. The present invention also provides a method for assessing the risk of neonatal biliary atresia. The present invention provides a biomarker for preparing products, methods or devices for assessing the risk of neonatal biliary atresia that can be used for clinical screening of patients at high risk of neonatal biliary atresia.

All of the technical solutions provided in the present invention for assessing the risk of neonatal biliary atresia can be used to determine whether to take further clinical diagnostic and therapeutic measures according to the results of the assessment. Based on the conceptions, methods, and data provided herein, the present invention enables early and rapid screening for neonatal biliary atresia.

The purpose of the present invention is to provide a method for quantitative detection of bilirubin in bilirubin-containing test samples, and particularly of each components of bilirubin. The purpose of the present invention is also to provide a kit suitable for the detection method. The purpose of the present invention is also to provide a device suitable for the detection method. The purpose of the present invention is also to provide the use of the detection method in the field of medical detection.

The present invention explores the association of bilirubin and its metabolites with the risk of newborns suffering from biliary atresia. The present invention confirms that bilirubin and its metabolites are associated with the risk of neonatal biliary atresia by determination of bilirubin and its metabolites in blood samples from both healthy newborns and newborns suffering from biliary atresia. In combination with bioinformatics methods, the levels of the biomarkers of the present invention are correlated with the risk of biliary atresia in the test subjects. As an example, bilirubin β-monoglucuronide and/or bilirubin γ-diglucuronide are used as biomarkers to detect the content of bilirubin β-monoglucuronide and/or bilirubin γ-diglucuronide in test samples from both healthy newborns and patients with neonatal biliary atresia to determine the cutoff value; when bilirubin β-monoglucuronide and/or bilirubin γ-diglucuronide in the test samples from test subjects is higher than the cutoff value in the same sample, it indicates that the test subject is at high risk of biliary atresia, suggesting the necessity for further clinical diagnosis and treatment.

The present invention provides biomarkers associated with neonatal biliary atresia, and further provides uses of the biomarkers in the assessment, screening and/or diagnosis of the risk of neonatal biliary atresia, and further provides tools and methods for assessing the risk of neonatal biliary atresia, and further particularly provides uses of the biomarkers in the preparation of diagnostic products for the risk assessment of neonatal biliary atresia, as well as related diagnostic products and instructions thereof.

The present invention describes the research processes and results of biomarker screening and validation. With the difference in the content of bilirubin and its components determined quantitatively in blood samples from normal newborns, newborns with biliary atresia, and newborns with cholestasis caused by other reasons, it is found that the values of quantitative detection for bilirubin β-monoglucuronide and/or bilirubin γ-diglucuronide can be used as indicators of risk assessment to distinguish among normal newborns, newborns with suspected biliary atresia and newborns with cholestasis caused by other reasons. Based on this creative research, the inventors further explore the use of the content of bilirubin β-monoglucuronide and/or bilirubin γ-diglucuronide as indicators for the early screening and diagnosis of clinical neonatal biliary atresia, thereby enabling rapid, convenient and efficient screening of biliary atresia after birth and promptly enabling further medical treatment for high-risk children.

The inventors propose bilirubin β-monoglucuronide and/or bilirubin γ-diglucuronide can be used to methods for rapid screening of clinical neonatal biliary atresia as biomarkers; and as an example, the inventors propose bilirubin β-monoglucuronide and/or bilirubin γ-diglucuronide as biomarkers can be used to prepare an appropriate diagnostic product for risk assessment of neonatal biliary atresia, wherein the diagnostic product uses bilirubin β-monoglucuronide and/or bilirubin γ-diglucuronide as quantitative detection indicators and comprises quantitative detection reagents for bilirubin β-monoglucuronide and/or bilirubin γ-diglucuronide, and optionally, can comprise reagents for processing a blood sample from a subject and other detection reagents.

Based on the discoveries and conceptions above, the first aspect of the present invention provides a use of conjugated bilirubin as a biomarker in the preparation of a diagnostic device for assessing the risk of neonatal biliary atresia, wherein the conjugated bilirubin is subject to a quantitative detection with the diagnostic device.

In some embodiments, the conjugated bilirubin comprises bilirubin β-monoglucuronide and/or bilirubin γ-diglucuronide.

In some embodiments, the diagnostic device is selected from medical devices, kits, test strips and detection devices; wherein the biological sample from a test subject is detected with the diagnostic device.

In some embodiments, the test subject is a newborn; and/or, the biological sample is a blood sample.

In some embodiments, the blood sample is selected from whole blood, plasma, serum and dried blood spots, such as dried blood spots.

In some embodiments, other detection indicator of the test subject can be optionally combined to assess the risk of neonatal biliary atresia with the diagnostic device, and the other detection indicator is selected from one or more of free bilirubin, biliverdin, γ-glutamyl transferase, MMP-7 and other indicators capable of directly or indirectly diagnosing neonatal biliary atresia.

In some embodiments, the biological sample is contained in an organic solvent dispersion system.

In some embodiments, the organic solvent of the organic solvent dispersion system is selected from one or more of methanol, ethanol, acetone, propylene glycol, acetonitrile, and a combination thereof; and/or, after the biological sample dispensed in the organic solvent dispersion system, the supernatant is collected for detection according to the detection method provided by the present invention after the biological sample is centrifuged.

In some embodiments, the quantitative detection method is liquid chromatography-tandem mass spectrometry.

In some embodiments, the organic solvent dispersion system contains a first stabilizer and a second stabilizer. The first stabilizer is selected from an antioxidant having a certain solubility in a non-polar medium; and the second stabilizer is selected from an antioxidant having a certain solubility in a polar medium. The weight ratio of the content of the first stabilizer and the second stabilizer in the organic solvent dispersion system is in the range of 1:(1 to 50).

In some embodiments, the weight ratio of the content of the first stabilizer and the second stabilizer in the organic solvent dispersion system is in the range of 1:(10 to 45); and/or, the first stabilizer is selected from one or more of butylated hydroxyanisole (BHA), butylated hydroxytoluene (BHT), propyl gallate (PG), tertiary butylhydroquinone (TBHQ), and a combination thereof; the second stabilizer is ascorbic acid.

In some embodiments, the weight ratio of the content of the first stabilizer and the second stabilizer in the organic solvent dispersion system is in the range of 1:(20 to 40); and/or, the first stabilizer is butylated hydroxytoluene (BHT) and the second stabilizer is ascorbic acid.

In some embodiments, the weight ratio of the content of the first stabilizer and the second stabilizer in the organic solvent dispersion system is in the range of 1:(30 to 40).

In some embodiments, the biological sample can be processed by the following manners: after the addition of taurine bilirubin as the internal standard, the organic solvent dispersion system containing the first stabilizer and the second stabilizer is added into the biological sample for shaking in the dark and centrifuging, and the supernatant is collected for detection.

In some embodiments, the organic solvent dispersion system containing the first stabilizer and the second stabilizer can be a methanol-acetonitrile mixture of BHT and ascorbic acid; as a specific embodiment, the weight ratio of the content of the first stabilizer and the second stabilizer in the system is in the range of 1:1-1:50, preferably 1:10-1:45, more preferably 1:20-1:40, further more preferably 1:30-1:40.

Preferably, the shaking and centrifugation are carried out at 0-10° C., optionally 0° C., 4° C., 10° C.; the condition for shaking is 1000-2000 rpm, preferably 1400-1500 rpm; the time for shaking is 10-30 minutes, preferably 15-25 minutes; the centrifugal force is 100000-250000 g and the time for centrifugation is 10-30 minutes.

In some embodiments, the diagnostic device may optionally combine other detection indicator of the test subject to assess the risk of neonatal biliary atresia, and the other detection indicator is selected from one or more of free bilirubin, biliverdin, γ-glutamyl transferase, MMP-7 and other indicators capable of directly or indirectly diagnosing neonatal biliary atresia.

The second aspect of the present invention provides a method for detection of the content of bilirubin, wherein the method for detection is a quantitative detection method of liquid chromatography tandem mass spectrometry. The “bilirubin” of the present invention refers to any one or more of free bilirubin, biliverdin, bilirubin β-monoglucuronide (BMG) and bilirubin γ-diglucuronide (BDG) in a test sample. When detecting the content, the aforementioned components can be determined separately or in combination, i.e. the content of bilirubin can be the content or the total content of any one or more of free bilirubin, biliverdin, bilirubin β-monoglucuronide and bilirubin γ-diglucuronide, or the sum thereof.

In some embodiments, in the liquid chromatography tandem mass spectrometry, the liquid chromatography is selected from high performance liquid chromatography, ultra high performance liquid chromatography and nanoliter liquid chromatography.

In some embodiments, the liquid chromatography is high performance liquid chromatography, and the column of the liquid chromatography is selected from C8 and C18 silica gel-packed columns. As an example, the column can be selected from Acquity BEH C18 (2.1*50 mm, 1.7 μm).

In some embodiments, the column temperature is at 35-45° C., the injection volume is 1-20 μL, with the flow rate as 0.2-0.6 mL/min.

In some embodiments, in the liquid chromatography tandem mass spectrometry, the composition of the mobile phase of the liquid chromatography is: aqueous phase A, selected from ultrapure water with a pH of 3-4.5; organic phase B, selected from one or more of acetonitrile, ethanol, methanol, propylene glycol, isopropanol, and a combination thereof.

In some embodiments, the pH is adjusted by a pH adjuster, wherein the pH adjuster is preferably selected from ammonium acetate-acetic acid, ammonium formate-formic acid, trifluoroacetic acid, and trichloroacetic acid buffer systems; as a preferable example, the pH adjuster is an ammonium acetate-acetic acid buffer system.

In some embodiments, the organic phase B is selected from one or more of acetonitrile, methanol, isopropanol, and a combination thereof; as a preferable example, the organic phase B includes acetonitrile, methanol, and isopropanol.

In some embodiments, gradient elution is adopted by the liquid chromatography; the following is one of the gradient elutions for exemplary purposes: 0 min, 95% mobile phase A and 5% mobile phase B; 0.5 min, 70% mobile phase A and 30% mobile phase B; 2.0 min, 5% mobile phase A and 95% mobile phase B; 4.0 min, 5% mobile phase A and 95% mobile phase B; 4.1 min, 95% mobile phase A and 5% mobile phase B; 5.0 min. 95% mobile phase A and 5% mobile phase B.

In some specific embodiments, the mass spectrometry in the liquid chromatography tandem mass spectrometry is selected from quadrupole mass spectrometry, time-of-flight mass spectrometry, ion hydrazine mass spectrometry and high-resolution orbital hydrazine mass spectrometry; the condition for the mass spectrometry and the mode settled for the qualitative and quantitative detection of mass spectrometry comprises: selecting electrospray ion source (ESI) and selecting ion scan mode based on the response of a target compound to be detected; selecting a multiple reaction monitoring method (MRM) and setting parameters for the multiple reaction monitoring mode.

In some specific embodiments, a preferable test sample is provided, wherein, the test sample of the detection method is dispensed in the system containing the first stabilizer and the second stabilizer.

The first stabilizer and the second stabilizer, the test sample and processing thereof are as described in the use of the first aspect of the present invention.

The third aspect of the present invention provides a stable quantitative detection reagent for bilirubin, wherein the quantitative detection reagent for bilirubin comprises a bilirubin standard, and the bilirubin standard is dispensed in a system containing a first stabilizer and a second stabilizer; the bilirubin standard comprises a standard of any one or more selected from free bilirubin, biliverdin, bilirubin β-monoglucuronide and bilirubin γ-diglucuronide.

In some embodiments, the first stabilizer and the second stabilizer are the first stabilizer and the second stabilizer as described in the use of the first aspect of the present invention.

In some embodiments, the system containing the first stabilizer and the second stabilizer is an organic solvent dispersion system containing butylated hydroxytoluene (BHT) and ascorbic acid.

In some embodiments, the organic solvent of the organic solvent dispersion system is selected from one or more of methanol, ethanol, acetone, propylene glycol, acetonitrile, and a combination thereof.

In some embodiments, the system containing a first stabilizer and a second stabilizer is a solid system, and the solid system comprises a solid carrier.

In some embodiments, the solid carrier is a filter paper sheet.

In some embodiments, the filter paper sheet is selected from analytical filter paper, qualitative analytical filter paper and slow quantitative ashless filter paper; and/or, when the filter paper sheet is used as a solid carrier, the filter paper sheet is pretreated as follows: the filter paper sheet is soaked in an organic solvent dispersion system containing a first stabilizer and a second stabilizer and then dried in the shade.

The preparation method of any detection reagent as described above, comprising dispensing the standard in the system.

The fourth aspect of the present invention provides a use of the stable quantitative detection reagent for bilirubin as described in the third aspect of the present invention in the preparation of a kit for detecting the content of bilirubin content or assessing the risk of biliary atresia, infant hepatitis syndrome, α1 antitrypsin deficiency disease or Alagille syndrome in a subject.

The fifth aspect of the present invention provides a kit for detecting the content of bilirubin or assessing the risk of neonatal biliary atresia, wherein the kit comprises the quantitative detection reagent for bilirubin as described in the third aspect of the present invention; the content of bilirubin is the content of bilirubin as described in the method for detection of the second aspect of the present invention.

In some embodiments, the kit comprises the standard and internal standard of any one or more of free bilirubin, biliverdin, bilirubin β-monoglucuronide and bilirubin γ-diglucuronide.

In some embodiments, the internal standard comprises a taurine bilirubin standard.

In some embodiments, the standard of any one or more of free bilirubin, biliverdin, bilirubin β-monoglucuronide and bilirubin γ-diglucuronide are dispensed in a system containing a first stabilizer and a second stabilizer, respectively.

In some embodiments, the system is selected from a liquid dispersion system containing the standard and a test strip carrying the standard; and/or, the first stabilizer and the second stabilizer are the first stabilizer and the second stabilizer as described in the first aspect of the present invention.

In some other embodiments, the system is a solid system, and the solid system is as described in the detection reagent of the third aspect of the present invention.

The detection method for bilirubin content and the kit for detecting bilirubin content or assessing the risk of neonatal biliary atresia provided by the present invention can be used for determining several types of bilirubin separately or simultaneously, and for quantitative detection of free bilirubin, biliverdin, bilirubin β-monoglucuronide and bilirubin γ-diglucuronide for any detection purpose. For an exemplary purpose, the detection method and the kit can be used for diagnosis, risk assessment and medicament efficiency assessment of any disease or condition sensitive to one or more of these detection indicators, such as medical-related diagnosis, risk assessment, etc. For example, these diseases or conditions are selected from neonatal biliary atresia, infant hepatitis syndrome, α1 antitrypsin deficiency disease and Alagille syndrome, etc.

The sixth aspect of the present invention provides a use of the kit as described in the fifth aspect of the present invention, wherein the use comprises assessing the risk of biliary atresia, infantile hepatitis syndrome, α1 antitrypsin deficiency disease and Alagille syndrome of a subject; preferably, the use further comprises the use instruction of the kit, and the use instruction is defined by the detection method as described in the second aspect of the present invention.

The seventh aspect of the present invention provides a method for assessing the risk of neonatal biliary atresia, using the kit as described in the fifth aspect of the present invention as the kit for assessing the risk of neonatal biliary atresia, comprising:

(1) Taking a newborn as a test subject to obtain the biological sample as described in the fifth aspect of the present invention as a test sample.

(2) Quantifying the expression level of a biomarker in the test sample with the detection method as described in the second aspect of the present invention; analyzing the expression level of the biomarker for risk assessment, and the analysis can be a comparison with the cutoff value of a quantitative detection, and the conclusion of the risk assessment can be used in assessing the risk of biliary atresia of the test subject is high or low.

The biomarker comprises bilirubin β-monoglucuronide and/or bilirubin γ-diglucuronide, and optionally in combination of other detection indicator of the test subject to assess the risk of neonatal biliary atresia, wherein the other detection indicator is selected from any one or more of free bilirubin, biliverdin, γ-glutamyl transferase, MMP-7 and other indicators capable of directly or indirectly diagnosing neonatal biliary atresia.

The quantitative detection method is any one of liquid chromatography-tandem mass spectrometry described above.

The test sample is selected from whole blood, plasma, serum and dried blood spots of the test subject.

(3) Analyzing the expression level of the biomarker for risk assessment, wherein the analysis can be a comparison with the cutoff value of a quantitative detection, and the conclusion of the risk assessment can be used in assessing the risk of biliary atresia of the test subject is high or low.

(4) Dividing the test subject into a high risk group and a low risk group based on the assessment conclusion, wherein the high risk group indicates the need for further clinical diagnosis to determine whether the test subject has biliary atresia.

In some preferable embodiments, the cutoff value of the quantitative detection in step (3) is a value determined by statistical analysis with the quantitative detection in step (2), wherein the expression level of the biomarker in the test sample of a healthy newborn and of the corresponding biomarker in the test sample of a newborn with biliary atresia is determined in advance.

The eighth aspect of the present invention provides a device for predicting the risk of neonatal biliary atresia by the expression level of a conjugated bilirubin, comprising:

(1) A module for receiving a test sample of a test subject.

(2) A module for detecting data on the expression level of a biomarker; wherein the biomarker comprises at least a conjugated bilirubin, wherein the conjugated bilirubin is selected from bilirubin β-monoglucuronide and/or bilirubin γ-diglucuronide, and optionally comprises free bilirubin, biliverdin, γ-glutamyl transferase, MMP-7 and other clinical indicator capable of diagnosing biliary atresia in the test sample.

(3) A module for generating a risk score based on inputting the expression level of a biomarker to a database, wherein the database comprises a control expression profile associated with the test sample and the method for detection; the control expression profile is derived in advance on the basis of the test sample and the method for detection, which may be expressed as a cutoff value for the biomarker detected; the risk assessment is performed by comparing the expression level of the biomarker in the test sample with the cutoff value of the high performance liquid chromatography tandem mass spectrometry as the method for detection, and the test subject is considered to be at high risk of biliary atresia when the expression level of the biomarker is higher than the cutoff value.

The device is selected from diagnostic product and analytical detection device.

The “diagnostic product” of the present invention can be medical instruments, kits, test strips and medical devices, etc. As known to a person of ordinary skill in the art, the definition of medical instruments, kits, etc. is related to the relevant provision of regulations, laws, and policies of governmental authorities, which varies in classifications and meanings in different countries and regions. The terms such as medical instruments, kits, test strips, etc. of the present invention are intended only to illustrate the form of use of the biomarkers of the present invention, and are not the meanings under the definition of governmental authorities; the diagnostic products can be medical products registered by the relevant governmental authorities, or products or product combinations used by a person of ordinary skill in the art in the manner or form of temporary application, as long as they are consistent with the purpose of the present invention.

The “test sample” described herein can include body fluid, blood, plasma, serum derived from the test subject, preferably include blood, plasma, serum samples. As an outstanding effect of the present invention, it has been found that the biomarkers provided by the present invention can be detected in a small amount of the test sample from the test subject, such as heel blood or fingertip blood, or dried blood spots prepared from heel blood or fingertip blood.

The “blood sample” of the present invention can be whole blood, plasma, serum, heel blood or fingertip blood collected from the test subject or dried blood spots routinely used in clinical practice, wherein the dried blood spots may be collected from the heel blood or fingertip blood of the test subject. The detection method of the present invention overcomes the limitations of conventional biochemical methods and enables the qualitative and quantitative determination of bilirubin with a small amount of the biological sample.

As one of the examples of the present invention, the present invention provides a kit for assessing the risk of neonatal biliary atresia, comprising quantitative detection reagents for bilirubin β-monoglucuronide, such as a quality control, an internal standard and an internal standard dilution. As an embodiment, the quality control comprises a quality control containing bilirubin β-monoglucuronide, the internal standard comprises a taurine bilirubin standard, and the internal standard dilution comprises a methanolic acetonitrile solution containing 2,6-Di-tert-butyl-4-methylphenol. The internal standard dilution may further comprise an aqueous solution containing ammonium acetate and acetic acid.

As one of the examples of the present invention, dried blood spots prepared from blood collected from the heel or fingertip of a newborn are used as the test sample. The subject may be considered to be at high risk of neonatal biliary atresia when the content of bilirubin β-monoglucuronide is greater than 2.5 μM, under the detection condition of the examples of the present invention. As one of the specific embodiments, the applicant uses the content of bilirubin monoglucuronide greater than 2.5 μM in the blood sample of the subject as the cutoff value under the detection condition of the examples, leading to rapid detection and assessment of the risk of neonatal biliary atresia. The Cutoff value in the example is a standard determined under the conditions such as the detection method, the detection condition and the standard. According to the teaching of the present invention, a person of ordinary skill in the art can screen and optimize the quantitative detection method for bilirubin β-monoglucuronide, including but not limited to high performance liquid chromatography techniques, such as gas chromatography mass spectrometry detection techniques. Under the teaching of the present invention, the cutoff value may be optimized and determined based on the detection method and detection condition for the risk assessment of neonatal biliary atresia. In the aforementioned screening of cutoff values of different detection methods, the detection method of the present invention can also be used as a reference and control, especially in the absence of clinical samples to determine cutoff values of other detection methods. Therefore, the use of the content of bilirubin β-monoglucuronide as a detection indicator to assess the risk of neonatal biliary atresia should be understood as falling within the scope enable for a person of ordinary skill in the art under the teachings of the present invention, regardless of the differences in the detection methods and cutoff values.

The Positive Progressive Effect of the Present Invention is that:

The technical solution proposed in the present invention can effectively assess the risk of neonatal biliary atresia and enables early screening of neonatal biliary atresia in clinical practice. In clinical use, newborns with a high risk of biliary atresia can be rapidly screened by quantitative determination of the content of the biomarkers in blood samples of the newborns. The biomarkers of the present invention, as markers for screening neonatal biliary atresia, may also be optionally combined with other detection indicators and detection manners to further assess the risk of neonatal biliary atresia, as well as optionally combined with other detection indicators and detection manners in the diagnostic process. The other detection indicators are selected from γ-glutamyl transferase, MMP-7 and clinical indicators.

The technical solution provided by the present invention enables the quantitative detection of each component of bilirubin separately or in combination. The qualitative and quantitative methods for bilirubin and its metabolites are established by using a liquid chromatography tandem mass spectrometer. In the research, the optimization of the instrumental analysis method, including liquid chromatography conditions, mass spectrometry detection conditions and mass spectrometry parameters optimization, and sample pre-treatment, enable accurate and rapid detection of the target substances qualitatively and quantitatively, specifically including the selection of liquid phase conditions, settlement of mass spectrometry conditions, preparation of standard solutions, optimization of analytical methods, drawing of standard curves, pre-treatment of the sample, and qualitative and quantitative detection of bilirubin and its metabolites in samples.

The present invention uses liquid chromatography tandem mass spectrometry to quantitatively detect the content of each component of bilirubin, which has the advantages of convenient sampling and reliable detection results, and has the characteristics of high accuracy and low limit of detection, compared with the traditional biochemical method. Meanwhile, it can simultaneously detect free bilirubin, biliverdin, bilirubin β-monoglucuronide and bilirubin γ-diglucuronide, and provides more indicators for scientific research, clinical diagnosis and assessment.

The technical solution proposed in the present invention may effectively assess the risk of neonatal biliary atresia and may enable early screening of neonatal biliary atresia in clinical practice. In clinical use, newborns with a high risk of biliary atresia can be rapidly screened by quantitative determination of the content of the biomarkers in blood samples of the newborns. The biomarkers of the present invention, as markers for screening neonatal biliary atresia, may also be optionally combined with other detection indicators and detection manners to further assess the risk of neonatal biliary atresia, as well as optionally combined with other detection indicators and detection manners in the diagnostic process. The other detection indicators are selected from γ-glutamyl transferase, MMP-7 and clinical indicators.

The present invention uses high performance liquid chromatography tandem mass spectrometry to quantitatively detect the content of bilirubin and its metabolites in blood samples as biomarkers of neonatal biliary atresia, which has the advantages of convenient sampling, high efficiency and reliable results. Blood samples may be selected from whole blood, plasma, serum and dried blood spots. The dried blood spots are collected from the heel blood or fingertip blood of the newborn. As one of the embodiments, the inventors explored the use of dried blood spots as a diagnostic product of the test sample. The convenience of dried blood spot sampling facilitates rapid screening for biliary atresia after birth and further facilitates the widespread promotion of rapid screening for biliary atresia after birth. From another aspect, even if re-sampling is required for repeating detection, it is particularly advantageous because of the mature technology of dried blood spot preparation, convenience of sampling, and minor or no trauma to the newborns. In addition, the present disclosure provides a quantitative detection method with a high performance liquid-mass spectrometry, which has the characteristics of high accuracy and low limit of detection compared with the traditional biochemical method.

The present invention for the first time proposes the use of the biomarker of the present invention as a biomarker for screening and risk assessment of neonatal biliary atresia, which can be a use to assess the risk of neonatal biliary atresia, and can be used clinically to screen the risk of neonatal biliary atresia at an early stage and further diagnosis, so as to strive for the best surgery time for pediatric patients with neonatal biliary atresia and improve the prognosis of surgery.

Compared with the prior art, the present invention has the advantages of simplicity and rapidity, flexibility in sample preparation, low limit of detection, good reproducibility and high sensitivity; and the sample preparation and detection method are simple and easy to implement, cost effective, and suitable for promotion and use.

All references cited herein are incorporated herein by reference in their entireties and used for all purposes to the extent that it is explicitly and separately indicated that each publication or patent or patent application is incorporated herein by reference in its entirety and used for all purposes. In addition, the CAS registry numbers cited herein are incorporated herein by reference in their entireties and used for all purposes to the extent that it is explicitly and separately indicated that each of such numbers is incorporated herein by reference in its entirety and used for all purposes.

Unless otherwise defined, all technical and scientific terms used herein have the same meaning as commonly understood by a person of ordinary skill in the art to which the present invention belongs. As used in this specification and the appended claims, the singular forms “a” and “the” include more than one of the objects referred to, unless the content clearly indicates a meaning different from this. For example, when referring to “component,” it includes the combination of one or more components, etc.

In order to make the purpose, the technical solution and the effect of the present invention clearer and more definite, the present invention is described in further detail hereinafter in combination with the accompanying drawings and examples. It should be understood that the specific examples described herein are intended only to explain the present invention and are not intended to limit it.

### BRIEF DESCRIPTION OF THE DRAWINGS

FIG. 1 is the liquid chromatography graph of bilirubin and its metabolites with the ammonium acetate-acetic acid system as mobile phase A. It can be seen from the figure that each target test subject can be separated effectively.

FIG. 2 is the liquid chromatography graph with ammonium formate-formic acid system as mobile phase A. It can be seen from the figure that each target test subject can be separated effectively.

FIG. 3 is the liquid chromatography graph with the trifluoroacetic acid system as mobile phase A. It can be seen from the figure that the target test subject can be separated effectively.

FIG. 4 is the standard curve prepared according to the quality control for the detection of the target components by high performance liquid chromatography tandem mass spectrometry.

FIG. 5 is the area under the ROC curve for each metabolite in distinguishing between newborns with biliary atresia from normal newborn controls.

FIG. 6 is a set of scatter plots showing the difference of levels of bilirubin β-monoglucuronide in test samples of newborns with biliary atresia and normal newborns, and the area under the ROC curve for bilirubin β-monoglucuronide in distinguishing newborns with biliary atresia from normal newborn controls.

FIG. 7 is a set of scatter plots showing the difference of levels of bilirubin β-monoglucuronide in test samples of newborns with biliary atresia and newborns with other cholestasis, and the area under the ROC curve for bilirubin β-monoglucuronide in distinguishing newborns with biliary atresia from newborns with other cholestasis.

FIG. 8 is the area under the ROC curve for the combination of bilirubin β-monoglucuronide and bilirubin γ-diglucuronide in distinguishing newborns with biliary atresia from normal newborn controls.

FIG. 9 is a set of scatter plots showing the difference of levels of bilirubin β-monoglucuronide in test samples of pediatric patients with infant hepatitis syndrome, pediatric patients withal antitrypsin deficiency disease, pediatric patients with Alagille syndrome and normal newborn controls, and the area under the ROC curve for bilirubin β-monoglucuronide in pediatric patients with infant hepatitis syndrome, pediatric patients with α1 antitrypsin deficiency disease, pediatric patients with Alagille syndrome from normal newborn controls.

### DETAILED DESCRIPTION OF THE EMBODIMENT

The major bilirubin in human blood include four currently known bilirubin components in serum, namely α-, β-, γ-, and δ-bilirubin (bilirubin IXα, Bα; bilirubin β-monoglucuronide, BMG; bilirubin γ-diglucuronide, BDG; δ-bilirubin, Bδ). Among them, α-bilirubin is unconjugated bilirubin (UCB) and β-, γ- and δ-bilirubin are conjugated bilirubin (CB). Under normal situations, α-bilirubin in hepatocytes has two main sources, one is uptaken from peripheral blood and transported into hepatocytes via organic anion transporting polypeptides (OATP1B1, OATP1B3) transporters on the basolateral membrane of hepatocytes, and the other is produced by oxidative degradation of hemoglobin in hepatocytes. In hepatocytes, 98%-99% of α-bilirubin is bound to one or two glucuronides, respectively, by the glucuronidation of UDP-glucuronosyltransferase 1A1 (UGT1A1), and is converted into β- or γ-bilirubin, which are excreted into the bile duct by multidrug resistance-associated protein 2 (MRP2) transporters on the capillary bile duct membrane. In cholestasis, especially obstructive cholestasis, bile secreted by hepatocytes cannot enter the bile ducts properly, resulting in the accumulation and elevation of peripheral blood bile acids (BA) and conjugated bilirubin. By measuring the content of conjugated bilirubin in the biological sample of the subject, it is of important significance for clinical diagnosis.

The inventors found that the prior art does not disclose the association of conjugated bilirubin and conjugated bilirubin-related components with neonatal biliary atresia, or explore their use as biomarkers to assess the risk of neonatal biliary atresia; nor does the prior art provide technical solutions or technical motivations for the use of conjugated bilirubin and conjugated bilirubin components in the diagnosis of neonatal biliary atresia.

The inventors have studied and explored the association of conjugated bilirubin and conjugated bilirubin-related metabolites with neonatal biliary atresia via extensive experiments, as well as their application in the field of risk assessment of clinical neonatal biliary atresia.

At present, the main biochemical methods for detection of free bilirubin and conjugated bilirubin are: diazonium salt modified J-G method, bilirubin oxidase method, chemical oxidation method, vanadate method and transcutaneous bilirubin measurement method. Both the diazonium and oxidase methods are easily interfered by lipids and hemolysis. At the same time, traditional biochemical methods require a large volume of blood, and is not operative clinically for newborns. Moreover, these methods have strict requirements for biological samples and can only detect conjugated bilirubin in serum or plasma. Transcutaneous bilirubin meters can only detect total bilirubin, but cannot distinguish free bilirubin and conjugated bilirubin.

The inventors first explored technical solutions for the quantitative detection of each component of bilirubin. In the examples, the qualitative and quantitative detection of each component of bilirubin was achieved by the research work, including selecting liquid phase conditions, setting mass spectrometry conditions, preparing standard solutions, optimizing analytical methods, drawing standard curves, and pre-treatment of samples. The following is illustrated by means of the examples.

The sources of the reagents used in the examples herein are as follows:
Bilirubin Conjugate, Ditaurate, Disodium Salt: Product No. 201102, purchased from Sigma-Aldrich, China
Biliverdin: Product No. 30891, purchased from Sigma-Aldrich, China
Free bilirubin: Product No. B4126, purchased from Sigma-Aldrich, China
Methanol: Product No. 900688, purchased from Sigma-Aldrich, China
Acetonitrile: Product No. 900667, purchased from Sigma-Aldrich, China
2,6-Di-tert-butyl-4-methylphenol (BHT): Product No. B1378, purchased from Sigma-Aldrich, China
Ammonium acetate: Product No. 73594, purchased from Sigma-Aldrich, China
Acetic acid: Product No. 45754, purchased from Sigma-Aldrich, China
Ammonium formate: Product No. 70221, purchased from Sigma-Aldrich, China
Formic acid: Product No. 5330020050, purchased from Sigma-Aldrich, China
Trifluoroacetic acid: Product No. 302031, purchased from Sigma-Aldrich, China
Trichloroacetic acid: Product No. T6399, purchased from Sigma-Aldrich, China
Bilirubin β-monoglucuronide, prepared in the applicant's laboratory, CAS No. 27071-67-6, molecular formula: C₃₉H₄₄N₄O₁₂, average molecular weight: 760.786, purity: ≥90%;
Bilirubin γ-diglucuronide, prepared in the applicant's laboratory, CAS No.: 17459-92-6, molecular formula: C₄₅H₅₂N₄O₁₈, average molecular weight: 936.921, purity: ≥90%.

### Example 1: Establishment and Optimization of Liquid Chromatography-Tandem Mass Spectrometry Method

In the present example, the process of establishing a qualitative and quantitative detection method for each component of bilirubin in biological samples based on liquid chromatography-tandem mass spectrometry is described for exemplary purposes, including selecting liquid phase conditions, setting mass spectrometry conditions, preparing standard solutions, optimizing the analytical method, and drawing the standard curve. The detection method of the present example has high sensitivity and high selectivity, and strong anti-interference ability, which may become an effective tool for qualitative and quantitative analysis of each component of bilirubin.

Bilirubin samples contain a variety of components. The inventors found that when the pH value of mobile phase A of liquid chromatography is 3-4.5, the major components in the bilirubin samples, such as free bilirubin, biliverdin, bilirubin β-monoglucuronide (monobilirubin) and bilirubin γ-diglucuronide (dibilirubin), can be well separated, and are suitable for a wider range of columns, especially C8 and C18 silica columns, and have good suitability for column temperature and injection volume.

The mass spectrometry can be quadrupole mass spectrometry, time-of-flight mass spectrometry, ion hydrazine mass spectrometry and high-resolution orbital hydrazine mass spectrometry; the conditions of the mass spectrometry and modes of the mass spectrometry set for the qualitative and quantitative detection include: selecting the electrospray ionization source (ESI) and selecting the ion scan mode according to the response of the target compounds to be detected; selecting the multiple reaction monitoring method (MRM) and setting the parameters of the multiple reaction monitoring mode.

### I. Study of Detection Conditions

Instruments and reagents: acetonitrile (chromatographic pure grade), methanol (chromatographic pure grade), isopropanol (chromatographic pure grade), ammonium acetate (chromatographic pure grade), acetic acid (chromatographic pure grade), manual or automatic pipettes (10-200 μL, 100-1000 μL), all are commercially available.

Liquid phase condition 1: mobile phase A: ultrapure water solution of pH 3.5 adjusted by ammonium acetate+acetic acid, mobile phase B: acetonitrile:methanol:isopropanol=8:1:1, column temperature: 40° C., column: Acquity BEH C18 (2.1*50 mm, 1.7 μm), injection volume: 5 μL.

Liquid phase condition 2: mobile phase A: ultrapure water solution of pH 4.5 adjusted by ammonium formate+formic acid, mobile phase B: acetonitrile:methanol:isopropanol=8:1:1, column temperature: 40° C., column: Acquity BEH C18 (2.1*50 mm, 1.7 μm), injection volume: 5 μL.

Liquid phase condition 3: mobile phase A: ultrapure water solution of pH 3 adjusted by ammonium trifluoroacetate, mobile phase B: acetonitrile:methanol:isopropanol=8:1:1, column temperature: 40° C., column: Acquity BEH C18 (2.1*50 mm, 1.7 μm), injection volume: 5 μL.

Instruments and reagents: acetonitrile (chromatographic pure grade), methanol (chromatographic pure grade), isopropanol (chromatographic pure grade), ammonium acetate (chromatographic pure grade), acetic acid (chromatographic pure grade), manual or automatic pipettes (10-200 μL, 100-1000 μL), all are commercially available.

Liquid phase conditions: mobile phase A: ultrapure water solution of pH 3-4.5 adjusted by 77 mg ammonium acetate+250 μL acetic acid, mobile phase B: acetonitrile:methanol:isopropanol=8:1:1, column temperature: 40° C., column: Acquity BEH C18 (2.1*50 mm, 1.7 μm), injection volume: 5 μL.

The procedure of high performance liquid chromatography is as shown in Table 1 below.

TABLE 1
Procedure of high performance liquid chromatography

| Time | Flow rate | Mobile | Mobile | |
|---|---|---|---|---|
| (min) | (mL/min) | phase A | phase B | Curve |
| 0 | 0.5 | 95 | 5 | 6 |
| 0.5 | 0.5 | 70 | 30 | 6 |
| 2.0 | 0.5 | 5 | 95 | 6 |
| 4.0 | 0.5 | 5 | 95 | 6 |
| 4.1 | 0.5 | 95 | 5 | 6 |
| 5.0 | 0.5 | 95 | 5 | 6 |

The mass spectrometry conditions were set up: as shown in Table 2, the electrospray ionization source (ESI) was selected, and the appropriate ion scan mode was sleeted according to the response of the compounds, the multiple reaction monitoring method (MRM) acquisition mode under positive ion mode was adopted, and the multiple reaction monitoring mode parameters were set up; the scan time was 0.4-4 min. The semi-automatic injection mode was adopted, and the standard solutions were injected into the ion source respectively. The corresponding parent ion peaks were selected, and their product ions were analyzed by secondary mass spectrometry to obtain fragment ion information and establish the MRM mass spectrometry detection method for the compounds.

TABLE 2
MRM mass spectrometry detection parameters
of bilirubin and its metabolites

| | | Parent | Product | | |
|---|---|---|---|---|---|
| No. | Compound name | ion (m/z) | ion (m/z) | CV | CE |
| 1 | Free bilirubin | 585.3 | 299.2 | 50 | 20 |
| 2 | Bilirubin β-monoglucuronide | 761.5 | 299.2 | 50 | 40 |
| 3 | Bilirubin γ-diglucuronide | 937.4 | 299.2 | 78 | 50 |
| 4 | Biliverdin | 583.25 | 297.17 | 60 | 34 |
| 5 | Taurine bilirubin | 799.35 | 406.2 | 18 | 46 |

During the analysis of mass spectrometry described in the above embodiments, the ion source temperature was 150° C., the capillary voltage was 3000 V, the desolventizing gas temperature was 500° C., the flow rate of desolventizing gas was 1000 L/h, and the collision gas was argon.

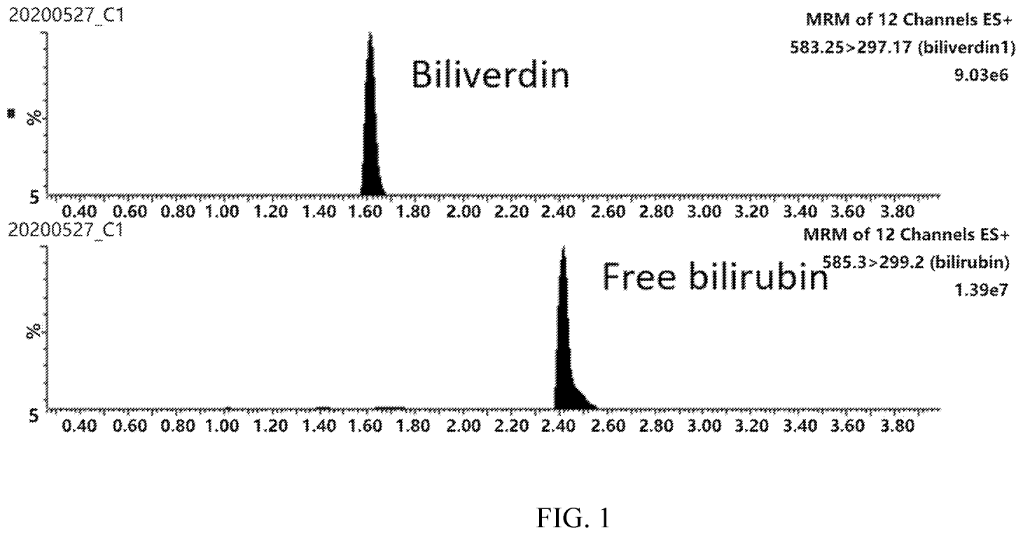

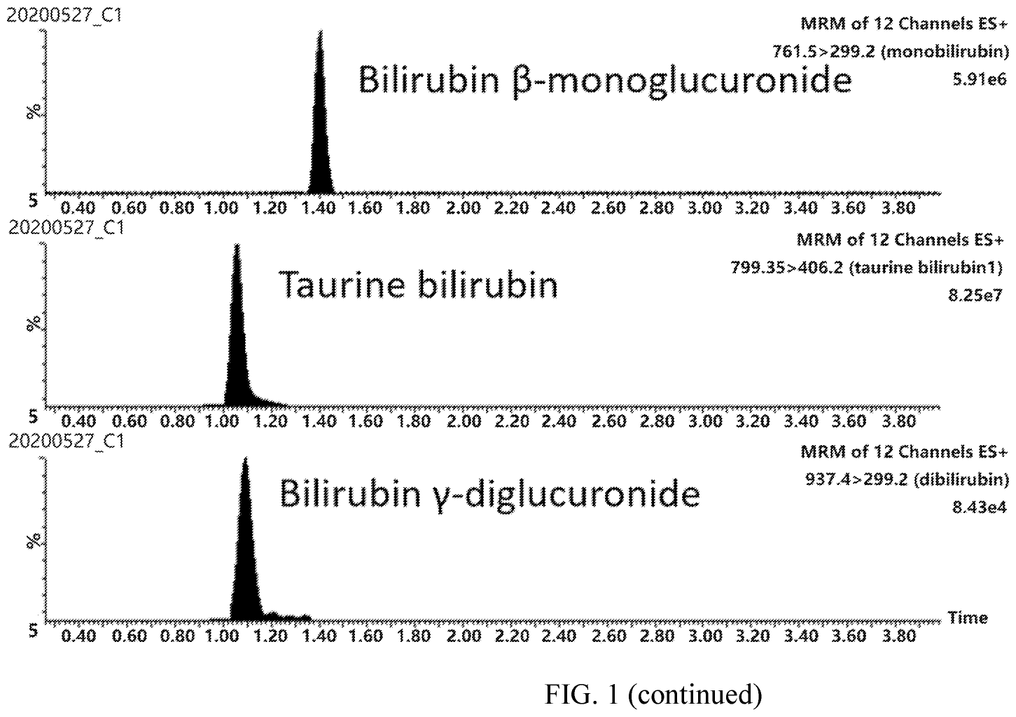

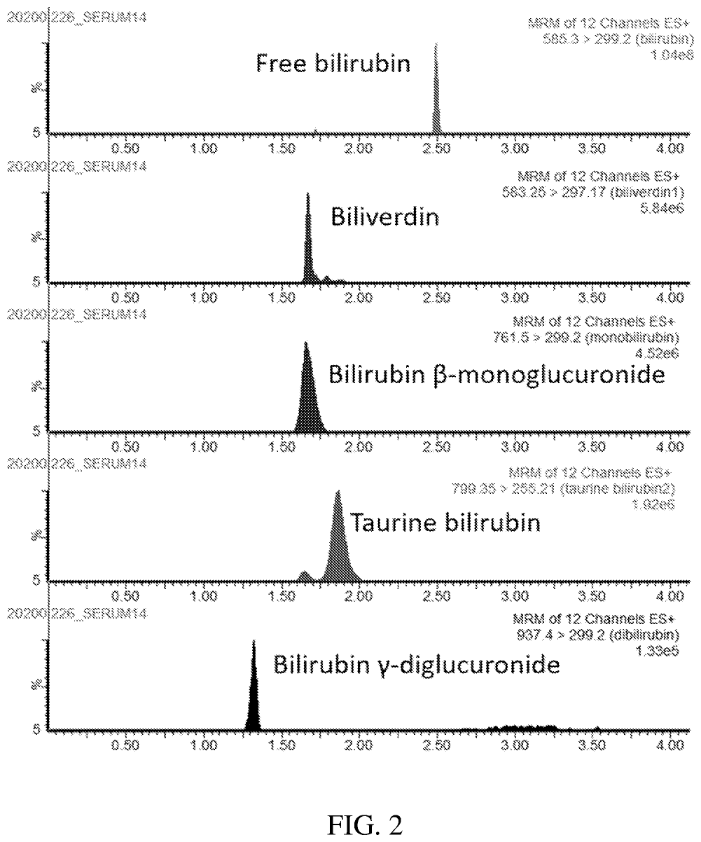

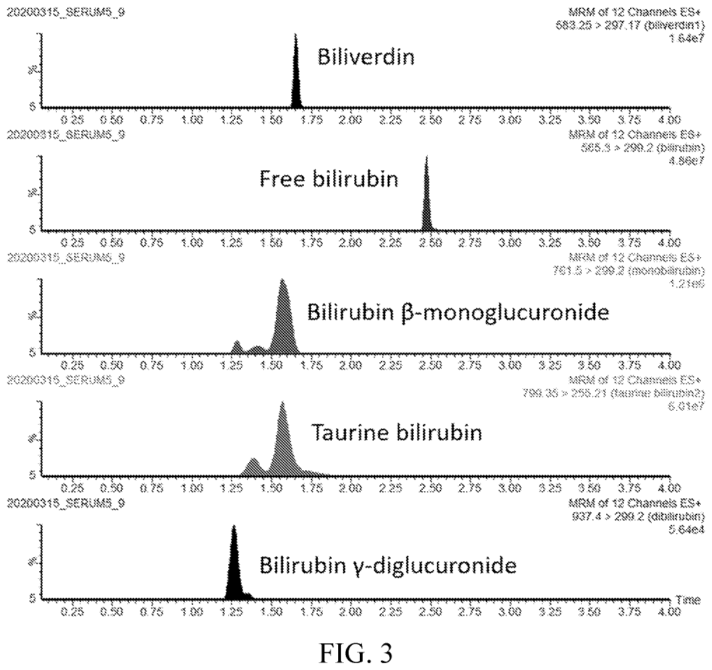

Under the above-described detection conditions with the three different liquid phase conditions, the typical chromatograms detected for each component of bilirubin are as shown in FIG. 1, FIG. 2 and FIG. 3, respectively.

### Preparation of Standard Solution

The above-described liquid phase condition 1 was used.

(1) Preparation of free bilirubin standard solution: an appropriate amount of free bilirubin was weighed, and chloroform was added to prepare the stock with a concentration of 1 mg/mL.

(2) Preparation of bilirubin β-monoglucuronide standard solution: an appropriate amount of bilirubin β-monoglucuronide was weighed, and 50% methanol solution was added to prepare the stock with a concentration of 1 mg/mL.

(3) Preparation of bilirubin γ-diglucuronide standard solution: an appropriate amount of bilirubin γ-diglucuronide was weighed, and 50% of methanol solution was added to prepare the stock with a concentration of 1 mg/mL.

(4) Preparation of biliverdin standard solution: an appropriate amount of biliverdin was weighed, and methanol was added to prepare the stock with a concentration of 1 mg/mL.

(5) Mixed standard stock: methanol containing 1% BHT was used to prepare the mixed standard, and each concentration of free bilirubin, bilirubin β-monoglucuronide, bilirubin γ-diglucuronide and biliverdin contained was 50 μmol/L.

(6) Mixed standard solution: methanol containing 1% BHT was used to dilute the mixed standard stock and the mixed standard solutions with a concentration of 20, 4, 1 and 0.2 μmol/L were obtained.

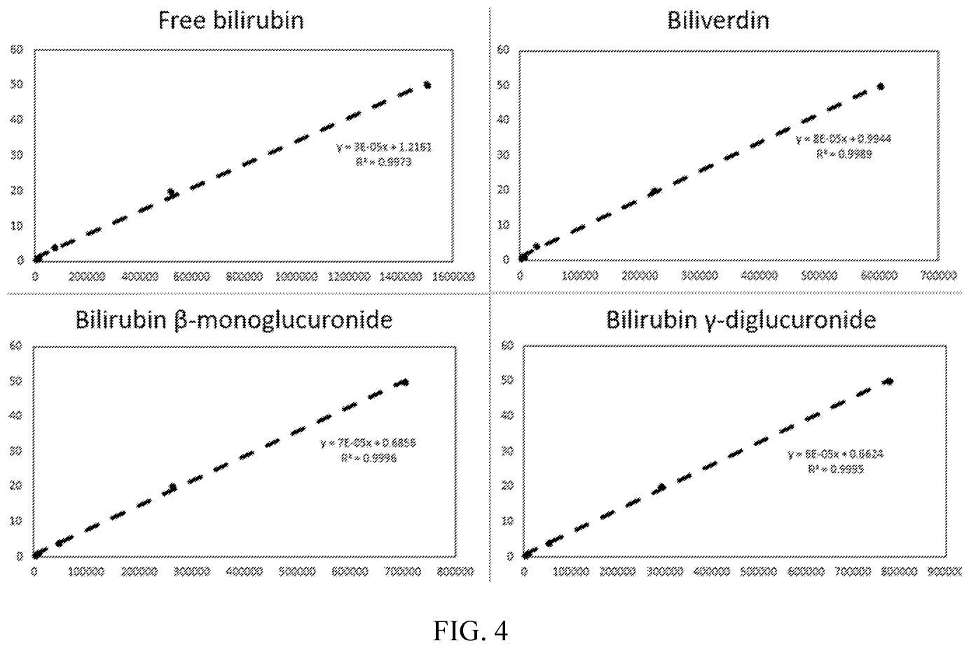

(7) The standard stock prepared in the above steps was diluted proportionally to prepare standard solution with the following concentrations, the injection volume was 1 μL, and the standard curve was drawn based on the peak area against the content of the target object. The obtained standard curve is as shown in FIG. 4.

### Example 2: Qualitative and Quantitative Determination of Bilirubin in Blood Samples

Sampling: serum, plasma and whole blood samples of 3 test subjects were collect, and each sample was divided into 3 groups, such as serum 01, serum 02, and serum 03, etc. 9 biological samples were obtained in total.

### Processing of Biological Sample:

Serum 01: 20 μL of sample was taken, 10 μL of taurine bilirubin was added as internal standard, then 80 μL of methanol:acetonitrile (1:1, v/v) solution containing 1 mg/mL BHT and 200 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Serum 02: 20 μL of sample was taken, 10 μL of taurine bilirubin was added as internal standard, then 80 μL of methanol:acetonitrile (1:1) solution containing 1 mg/mL BHT and 175 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Serum 03: 20 μL of sample was taken, 10 μL of taurine bilirubin was added as internal standard, then 80 μL of methanol:acetonitrile (1:1) solution containing 1 mg/mL BHT and 204 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Plasma 01: 20 μL of sample was taken, 10 μL of taurine bilirubin was added as internal standard, then 80 μL of methanol:acetonitrile (1:1) solution containing 1 mg/mL BHT and 180 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Plasma 02: 20 μL of sample was taken, 10 μL of taurine bilirubin was added as internal standard, then 80 μL of methanol:acetonitrile (1:1) solution containing 1 mg/mL BHT and 160 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Plasma 03: 20 μL of sample was taken, 10 μL of taurine bilirubin was added as internal standard, then 80 μL of methanol:acetonitrile (1:1) solution containing 1 mg/mL BHT and 170 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Whole blood 01: 20 μL of sample was taken, 10 μL of taurine bilirubin was added as internal standard, then 80 μL of methanol:acetonitrile (1:1) solution containing 1 mg/mL BHT and 200 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Whole blood 02: 20 μL of sample was taken, 10 μL of taurine bilirubin was added as internal standard, then 80 μL of methanol:acetonitrile (1:1) solution containing 1 mg/mL BHT and 206 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

Whole blood 03: 20 μL of sample was taken, 10 μL of taurine bilirubin was added as internal standard, then 80 μL of methanol:acetonitrile (1:1) solution containing 1 mg/mL BHT and 190 mmol/L ascorbic acid was added, and shaking for 20 min at 1450 rpm at 10° C. in the dark. It was centrifuged at 18,000 g for 20 min at 4° C. 60 μL of supernatant was taken and pipetted into a 96-well plate as the test sample for detection with high performance liquid chromatography tandem mass spectrometry.

The above treated samples were injected and detected according to the LC-MS/MS conditions in Example 1; the liquid phase condition 1 was selected and the sample was detected with internal standard for calibration and external standard for quantification. The detection results are shown in Table 3 below.

TABLE 3
Measurement indicators and measurement results

| | Content (μmol/L) | | | |
|---|---|---|---|---|
| | | | Bilirubin β- | |
| Sample | | Free | mono- | Bilirubin γ- |
| Numbering | Biliverdin | bilirubin | glucuronide | diglucuronide |
| Serum 01 | 1.021 | 22.26 | 6.721 | 0.398 |
| Serum 02 | 0.929 | 23.33 | 6.585 | 0.417 |
| Serum 03 | 0.979 | 22.11 | 6.64 | 0.388 |
| Plasma 01 | 2.353 | 4.251 | 10.07 | 1.379 |
| Plasma 02 | 2.464 | 4.047 | 8.951 | 1.406 |
| Plasma 03 | 2.361 | 4.034 | 9.726 | 1.392 |
| Whole blood 01 | 0.22 | 7.871 | 1.537 | 0.168 |
| Whole blood 02 | 0.245 | 7.741 | 1.608 | 0.182 |
| Whole blood 03 | 0.223 | 7.997 | 1.492 | 0.185 |

### Example 3: Qualitative and Quantitative Determination of Bilirubin Components in Dried Blood Spots

### Preparation of Dried Blood Spot Sample

Dried blood spot samples were prepared by collecting fingertip blood from newborns within 4 days of birth. The steps were as follows:

Pretreatment of filter paper sheets: 903 filter paper sheets were repeatedly soaked in ethanol solution containing 1 mg/mL BHT and 200 mmol/L ascorbic acid for 2 min, taken out and drained. Then the filter paper sheets were naturally dried in the shade for use.

Preparation: an appropriate amount of dilution was added to a suitable test tube. A micropipette was taken and connected to the latex tip, and the connection was checked for air leakage, or a disposable micropipette (siphon principle), blood collection needle, 75% ethanol or iodophor, cotton swabs, and etc. were taken for later use.

Massage: the central part of the ulnar side of the fingertip of the left ring finger where there are more muscles was gently massaged, so that the local tissue is naturally congested. The side of the finger or the fingertip were avoided using.

Disinfection: the blood collection site was wiped with 75% ethanol or iodophor swab and left to naturally dry.

Needle prick: the blood collection site was fixed with the thumb, index finger and middle finger of the left hand, so that the skin and subcutaneous tissue are taut, and a disposable sterile blood collection needle was held by the right hand pricked from the ventral ulnar side of the fingertip to a depth of 2-3 mm, and the needle was withdrawn immediately.

Blood swabbing: after the blood flows out naturally, the first drop of blood was wiped away with a sterile dry cotton ball (cotton swab).

Blood collection: disposable micropipette was used to collect blood or the blood was dropped on filter paper sheet, then sterile dry cotton ball (cotton swab) was used to press the wound to stop bleeding. Use the left hand to apply slight pressure from the distal end of the blood collection site to the finger end to make the blood flow out if there is poor blood flow.

A label for patient identification was made immediately after collecting the sample, so as to avoid confusion.

The sample was dried in a cool place in the dark for 4 hours, put into an aluminum foil bag and stored at 4° C.

### Pre-Treatment of Dried Blood Spot Samples

Three dried blood spots were collected from the dried blood spot with a perforated sampler, and 20 μL of pure water was added, shaken with a vortex at 1450 rpm, 10° C. for 20 min. 10 μL of internal standard (taurine bilirubin) and 80 μL of methanol:acetonitrile (1:1) solution containing 1 mg/mL 1 BHT and 200 mmol/L ascorbic acid was added, followed by continued shaking with a vortex at 1450 rpm, 10° C. for 20 min. The sample was centrifuged at a centrifugal force of 18000 g, 4° C. for 20 min. 60 μL of supernatant was pipetted into a 96-well plate and subject to sample injection.

### Determination by High Performance Liquid Chromatography-Tandem Mass Spectrometry

The sample was injected and detected according to the LC-MS/MS conditions in Example 1; the liquid phase condition 1 was selected and the sample was detected with internal standard for calibration and external standard for quantification. The determined content was as shown in Table 4 below, and the methodological parameters were as shown in Table 5.

TABLE 4
Measurement indicators and measurement results of content

| | Measurement indicators and measurement results (μmol/L) | | | |
|---|---|---|---|---|
| Sample | | Free | Bilirubin β- | Bilirubin γ- |
| Number | Biliverdin | bilirubin | monoglucuronide | diglucuronide |
| Dried blood | 0.601 | 12.08 | 0.988 | 0.212 |
| spot 01 | | | | |
| Dried blood | 0.663 | 12.15 | 1.014 | 0.214 |
| spot 02 | | | | |
| Dried blood | 0.620 | 11.76 | 1.095 | 0.203 |
| spot 03 | | | | |

TABLE 5
Methodological parameters

| | Methodological parameters of dried blood spot samples | | | | |
|---|---|---|---|---|---|
| | Limit of | Limit of | Linearity | | Reproducibility |
| Detection | detection | quantification | range | | (relative |
| indicators | (μmol/L) | (μmol/L) | (μmol/L) | R² | deviation, %) |
| Biliverdin | 0.1 | 0.5 | 0.5-50 | 0.994611 | 4.13 |
| Free bilirubin | 0.1 | 0.5 | 0.5-50 | 0.997855 | 1.41 |
| Bilirubin β- | 0.05 | 0.2 | 0.2-50 | 0.999581 | 4.41 |
| monoglucuronide | | | | | |
| Bilirubin γ- | 0.05 | 0.2 | 0.2-50 | 0.993738 | 2.28 |
| diglucuronide | | | | | |

### Example 4: Stable Quantitative Detection Reagent for Bilirubin

The present invention provides a stable quantitative detection reagent for bilirubin, comprising a bilirubin standard. The bilirubin standard is dispensed in a system containing a first stabilizer and a second stabilizer; the bilirubin comprises one or more of free bilirubin, biliverdin, bilirubin β-monoglucuronide and bilirubin γ-diglucuronide. The purpose of this example is to exemplarily provide a stable quantitative detection reagent for bilirubin, and to investigate the preparation method and stability thereof.

### Preparation of Stable Quantitative Detection Reagent for Bilirubin

Pre-treatment of filter paper sheets: analytical filter paper sheets were repeatedly soaked in ethanol solution containing 1 mg/mL BHT and 200 mmol/L ascorbic acid for 2 min, removed and drained. Then the filter paper sheets were naturally dried in the shade and stored at 4° C.

Preparation of quantitative detection reagents for bilirubin: free bilirubin, biliverdin, bilirubin β-monoglucuronide and bilirubin γ-diglucuronide were accurately weighed and dissolved in ethanol, respectively. The solutions were added dropwise onto the filter paper sheets with disposable micropipettes, dried in a cool place away from light for 4 hours, placed in an aluminum foil bag and stored at 4° C.

The samples were injected and detected according to the LC-MS/MS conditions in Example 1; liquid phase condition 1 was selected and the sample was detected with internal standard for calibration and external standard for quantification. The detection results were as shown in the table below; the samples which had been stored for the following days were quantified and the results are shown in Table 6.

TABLE 6

| | Stability of dried blood spot samples stored at 4° C. (mean ± standard deviation) | | | | | |
|---|---|---|---|---|---|---|
| Detection indicator | 0 day | 15 days | 30 days | 45 days | 60 days | 90 days |
| Bilirubin | 0.628 ± 0.025 | 0.638 ± 0.046 | 0.659 ± 0.074 | 0.633 ± 0.023 | 0.694 ± 0.027 | 0.828 ± 0.065 |
| Free bilirubin | 11.99 ± 0.17 | 11.62 ± 0.26 | 11.25 ± 0.34 | 11.57 ± 0.29 | 11.02 ± 0.33 | 10.62 ± 0.19 |
| Bilirubin β- | 1.032 ± 0.046 | 1.036 ± 0.056 | 1.094 ± 0.078 | 0.961 ± 0.052 | 1.004 ± 0.068 | 0.905 ± 0.019 |
| monoglucuronide | | | | | | |
| Bilirubin γ- | 0.209 ± 0.005 | 0.215 ± 0.006 | 0.203 ± 0.006 | 0.197 ± 0.004 | 0.201 ± 0.002 | 0.193 ± 0.006 |
| diglucuronide | | | | | | |

### Example 5: Kit for Bilirubin Quantitative Detection

The present invention provides a kit for bilirubin quantitative detection. The detection indicators for the kit comprises the content or the total content of any one or more of free bilirubin, biliverdin, bilirubin β-monoglucuronide and bilirubin γ-diglucuronide, or the sum thereof. The purpose of this example is to exemplarily provide a kit for bilirubin quantitative detection.

The kit comprising a bilirubin standard. The bilirubin standard is dispensed in a system containing a first stabilizer and a second stabilizer; the bilirubin comprises one or more of free bilirubin, biliverdin, bilirubin β-monoglucuronide and bilirubin γ-diglucuronide. The purpose of this example is to exemplarily provide a stable quantitative detection reagent for bilirubin, and to investigate the preparation method and stability thereof.

The composition of the kit of this example is as shown in Table 7.

TABLE 7

| Name | Main components |
|---|---|
| Quality control | Filter paper containing bilirubin standard |
| Internal standard | Taurine bilirubin standard |
| Internal standard dilution | Methanol:acetonitrile solution containing |
| | 2,6-Di-tert-butyl-4-methylphenol |

Herein, the filter paper containing bilirubin standard was prepared according to the following method:

The analytical filter paper sheets were repeatedly soaked in ethanol solution containing 1 mg/mL BHT and 200 mmol/L ascorbic acid for 2 min, removed and drained. Then the filter paper sheets were naturally dried in the shade and stored at 4° C. Free bilirubin, biliverdin, bilirubin β-monoglucuronide and bilirubin γ-diglucuronide were accurately weighed and dissolved in ethanol, respectively. The solutions were added dropwise onto the filter paper sheets with a disposable micropipette, dried in a cool place for 4 hours away from light, placed in an aluminum foil bag and stored at 4° C.

### Example 6: Applying the Detection Method of the Present Invention to the Screening of Neonatal Biliary Atresia

The technical solution provided by the present invention enables the quantitative detection method for bilirubin. The quantitative detection method for bilirubin and the kit for bilirubin quantitative detection may be used to determine several types of bilirubin separately or simultaneously, and for quantitative determination of free bilirubin, biliverdin, bilirubin β-monoglucuronide and bilirubin γ-diglucuronide for any detection purpose. The detection method and the kit can be used for diagnosis, risk assessment and medicament efficiency assessment of any disease or condition sensitive to one or more of these detection indicators, such as medical-related diagnosis, risk assessment, etc.

This example is illustrated by the technical solution of the present invention applied to the screening of neonatal biliary atresia; similarly, it can be applied to the screening, diagnosis, risk assessment and monitoring of the effectiveness of therapeutic drugs for other diseases or conditions that may cause changes in bilirubin composition. For example, these diseases or conditions are selected from neonatal biliary atresia, infant hepatitis syndrome, α1 antitrypsin deficiency disease, Alagille syndrome, etc. This example collected dried blood spots at birth from 39 normal newborns, 10 dried blood spots at birth from children with infant hepatitis syndrome, 9 dried blood spots from children with α1 antitrypsin deficiency disease, and 5 dried blood spots from children with Alagille syndrome.

This example investigated the content of bilirubin and its metabolites in blood samples from both normal newborns and newborns with biliary atresia. In this example, blood samples were collected from 39 clinical patients with neonatal biliary atresia and 300 normal newborn controls. The blood samples collected were dried blood spots prepared within 4 days of birth.

### Preparation of Dried Blood Spot Sample

Pretreatment of filter paper sheets: 903 filter paper sheets were repeatedly soaked in ethanol solution containing 1 mg/mL BHT and 200 mmol/L ascorbic acid for 2 min, taken out and drained. Then the filter paper sheets were naturally dried in the shade for use.

Preparation: an appropriate amount of dilution was added to a suitable test tube. A micropipette was taken and connected to the latex tip, and the connection was checked for air leakage, or a disposable micropipette (siphon principle), blood collection needle, 75% ethanol or iodophor, cotton swabs, and etc. were taken for later use.

Massage: the central part of the ulnar side of the fingertip of the left ring finger where there are more muscles was gently massaged, so that the local tissue is naturally congested. The side of the finger or the fingertip were avoided using.

Disinfection: the blood collection site was wiped with 75% ethanol or iodophor swab and left to naturally dry.

Needle prick: the blood collection site was fixed with the thumb, index finger and middle finger of the left hand, so that the skin and subcutaneous tissue are taut, and a disposable sterile blood collection needle was held by the right hand pricked from the ventral ulnar side of the fingertip to a depth of 2-3 mm, and the needle was withdrawn immediately.

Blood swabbing: after the blood flows out naturally, the first drop of blood was wiped away with a sterile dry cotton ball (cotton swab).

Blood collection: disposable micropipette was used to collect blood or the blood was dropped on filter paper sheet, then sterile dry cotton ball (cotton swab) was used to press the wound to stop bleeding. Use the left hand to apply slight pressure from the distal end of the blood collection site to the finger end to make the blood flow out if there is poor blood flow.

A label for patient identification was made immediately after collecting the sample, so as to avoid confusion.

The sample was dried in a cool place in the dark for 4 hours, put into an aluminum foil bag and stored at 4° C.

### Pre-Treatment of Dried Blood Spot Samples

Three dried blood spots were collected from the dried blood spot with a perforated sampler, 20 μL of pure water was added, shaken with a vortex at 1450 rpm, 10° C. for 20 min, 10 μL of internal standard (taurine bilirubin) and 80 μL of methanol:acetonitrile (1:1) solution containing 1 mg/mL 1 BHT and 200 mmol/L ascorbic acid was added, followed by continued shaking with a vortex at 1450 rpm, 10° C. for 20 min. The sample was centrifuged at a centrifugal force of 18000 g, 4° C. for 20 min. 60 μL of supernatant was pipetted into a 96-well plate and subject to sample injection.

### Determination by High Performance Liquid Chromatography-Tandem Mass Spectrometry

The sample was injected and detected according to the LC-MS/MS conditions in Example 1; the liquid phase condition 1 was selected and the sample was detected with internal standard for calibration and external standard for quantification. The result of determination shows that, in infants with biliary atresia, the mean value of bilirubin β-monoglucuronide is 28 μmol/L, the maximum value is 82 μmol/L, and the minimum value is 2.9 μmol/L; while in normal infants, the mean value of bilirubin β-monoglucuronide is 1.25 μmol/L, the maximum value is 3.10 μmol/L, and the minimum value is 0.02 μmol/L. Taking bilirubin β-monoglucuronide as the diagnostic indicator, when the cutoff value is 2.5 μmol/L, the resulting sensitivity of diagnosing biliary atresia with bilirubin β-monoglucuronide is 100% and the specificity is 95.2%.

In addition, the sensitivity of diagnosing biliary atresia with bilirubin γ-diglucuronide is 85% and the specificity is 95%. The sensitivity of diagnosing biliary atresia with the combination of bilirubin β-monoglucuronide and bilirubin γ-diglucuronide is 99% and the specificity is 95%.

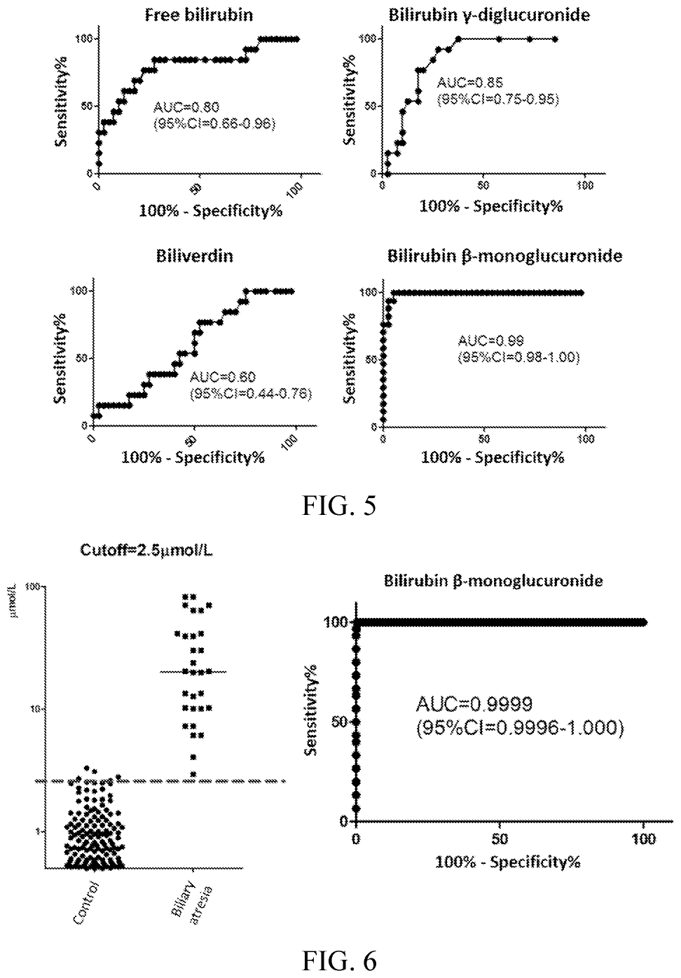

Therefore, the upregulation of bilirubin β-monoglucuronide and bilirubin γ-diglucuronide is promising to be a characteristic detection indicator for neonatal biliary atresia, as shown in FIG. 5, FIG. 6, and FIG. 8.

FIG. 5 shows area under the ROC curve of each metabolite distinguishing newborns with biliary atresia and normal newborn controls.

FIG. 6 is a set of scatter diagram, showing the difference of levels of bilirubin β-monoglucuronide between the test samples of newborns with biliary atresia and normal neonates.

FIG. 8 shows area under the ROC curve of the combination of bilirubin β-monoglucuronide and bilirubin γ-diglucuronide distinguishing between newborns with biliary atresia and normal newborn controls.

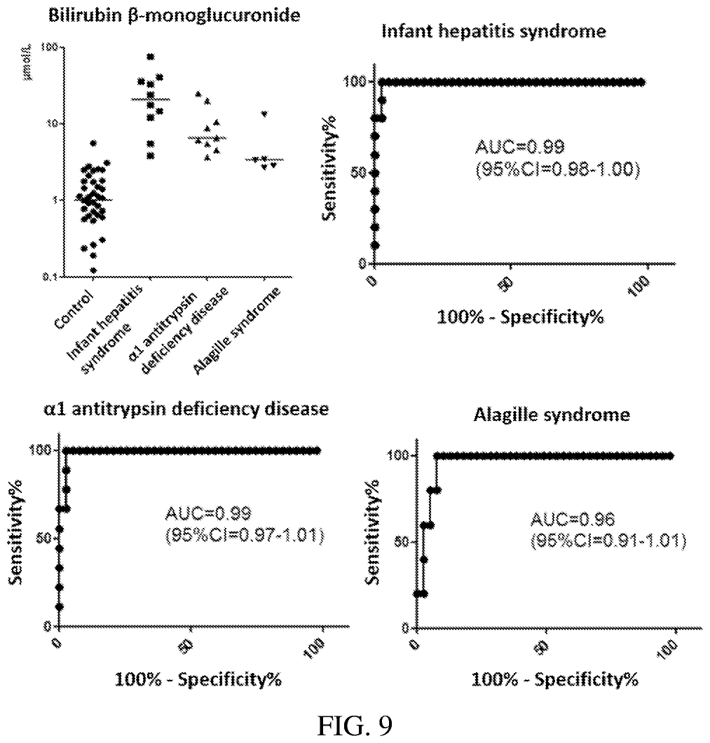

In infant hepatitis syndrome, α1 antitrypsin deficiency disease, and Alagille syndrome, the sensitivity of diagnosing infant hepatitis syndrome with bilirubin β-monoglucuronide is 99% and the specificity is 95%; the sensitivity of diagnosing α1 antitrypsin deficiency disease with bilirubin β-monoglucuronide is 99% and the specificity is 95%; the sensitivity of diagnosing Alagille syndrome with bilirubin β-monoglucuronide is 96% and the specificity is 95%. The results are shown in FIG. 9.

### Example 7: Distinguishing Patients with Biliary Atresia and Patients with Other Cholestatic Diseases

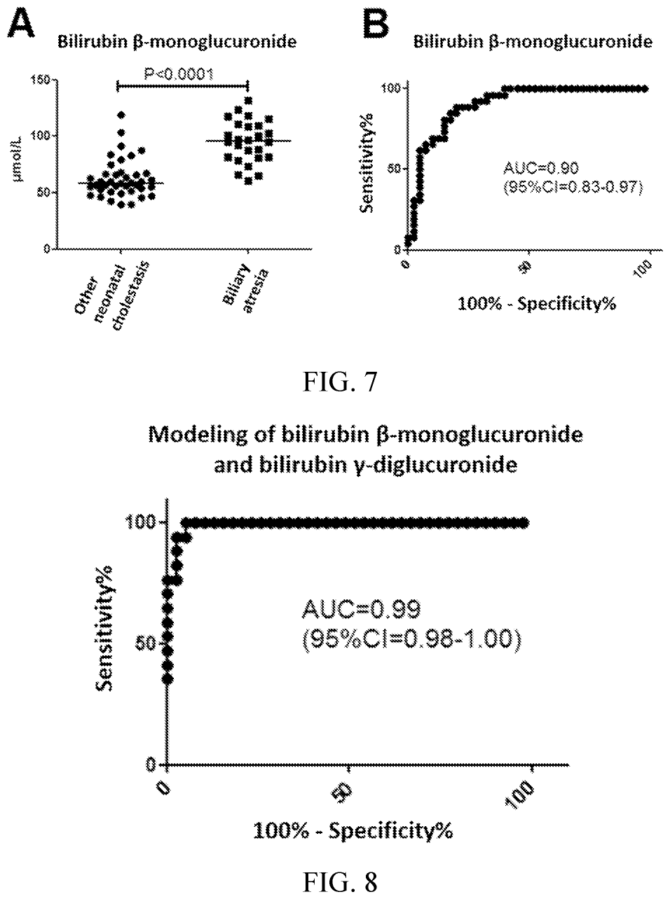

In this example, serum samples were collected from 26 newborns with biliary atresia and 40 newborns with cholestasis caused by other reasons. The content of bilirubin β-monoglucuronide in the serum samples were detected according to the high performance liquid chromatography tandem mass spectrometry method of Example 1. The detection results show that the content of bilirubin β-monoglucuronide in children with biliary atresia is significantly higher than that in other cholestatic infants (FIG. 7, P<0.0001); its area under the ROC curve reaches 0.90 (95% CI=0.83-0.97), with a cutoff value >75 μmol/L. Its sensitivity reaches 84.6% (65.1%-95.6%) and the specificity reaches 82.5% (67.2%-92.7%) under the detection condition of Example 1, thus bilirubin β-monoglucuronide may be used as a preliminary biomarker to distinguish biliary atresia from other infantile cholestasis.

### Example 8: Bilirubin β-Monoglucuronide as a Biomarker for the Preparation of a Diagnostic Tool

It can be found in the above examples that quantitative detection for bilirubin β-monoglucuronide can be used as an indicator to distinguish normal newborns, newborns with biliary atresia, and newborns with cholestasis caused by other reasons. Based on this creative research, the purpose of the present example is to exemplarily provide a use of bilirubin β-monoglucuronide content as an indicator for the early screening and diagnosis of clinical neonatal biliary atresia. A rapid, convenient, efficient and relatively accurate screening of biliary atresia after the birth of a newborn would be of great importance for children with high risk to take further medical treatment immediately.

To this end, the inventors propose, based on the purpose of application, that bilirubin β-monoglucuronide may be used as a biomarker for the preparation of a suitable tool. The tool uses bilirubin glucuronide as a biomarker and determine the risk of biliary atresia of the test subject is high or low according to its expression level. The tool may be a diagnostic product or an analytical test product. As a specific example, this example provides a kit as an example of a diagnostic product, which comprises quantitative detection reagents for bilirubin β-monoglucuronide, and optionally, may further comprises quantitative detection reagents for other metabolic components of bilirubin, and further comprises reagents for processing a blood sample from a subject, and may further comprises detection reagents for use in high performance liquid chromatography tandem mass spectrometry detection methods, such as internal standards, internal standard dilutions, mobile phase reagents for use in high performance liquid chromatography.

The inventors explore the preferred embodiment, wherein the kit uses dried blood spots as the test sample. The convenience of dried blood spot sampling facilitates rapid screening for biliary atresia after birth and further facilitates the widespread promotion of rapid screening for biliary atresia at an early age after birth. From another aspect, even if re-sampling is required for repeating detection, it is particularly advantageous because of the mature technology of dried blood spot preparation, convenience of sampling, and minor or no trauma to the newborns, even if repeated detection is required.

The kit usually includes at least a container for storing the standards. This container may be single-compartment or multi-compartment. For example, this container may be a multi-well plate (e.g., a 96-well plate), or other similar container. In some kits, this container is suitable for biomarkers used in the detection. In some kits, this container may be used for the detection of standard or for the instrumental analysis of biomarkers in the sample to be tested, such as high performance liquid chromatography tandem mass spectrometry analysis.

The following kit is one of the specific embodiments:

TABLE 8
Kit composition

| Component | | |
|---|---|---|
| Function | Component | Quantity |
| Quality control | Filter paper containing a certain | 1 pc |
| | concentration of bilirubin | |
| | β-monoglucuronide | |
| Internal standard | Taurine bilirubin standard | 1 pc |
| Internal standard | Methanol:acetonitrile solution containing | 10 mL/ |
| dilution | 2,6-Di-tert-butyl-4-methylphenol | pc × 1 pc |

### Example 9: Use of the Kit

The present invention further investigates and validates the use of the diagnostic kit.

In this Example, the inventors, according to the method of Example 1, collected dried blood spots prepared within 4 days of birth from 30 patients who have been clinically diagnosed with neonatal biliary atresia and from 562 normal newborns. In combination with the detection method in Example 1, the content of bilirubin β-monoglucuronide in the dried blood spots was detected with the reagents included in the kit of Example 3.

The detection results are shown in Table 9 below, when cutoff >2.5 μmol/L (A of FIG. 7), the sensitivity reaches 100% (88.43%-100%) and the specificity reaches 98.74% (97.42%-99.49%) (B of FIG. 7). It can be seen that it is reliable to use the content of bilirubin β-monoglucuronide in neonatal dried blood spots as a marker for the risk assessment of neonatal biliary atresia, and the kit may be used for risk assessment of neonatal biliary atresia.

TABLE 9

| Cutoff value | Sensitivity (95% CI) | Specificity (95% CI) |
|---|---|---|
| >2.5 μmol/L | 100% (88.43%-100%) | 98.74% (95.42%-99.49%) |

The above described is only a preferred embodiment of the present invention, and it should be noted that, for a person of ordinary skill in the art, several improvements and supplements can be made without departing from the method of the present invention, and these improvements and supplements should also be considered as the scope of protection of the present invention.

1. A method for assessing the risk of neonatal biliary atresia, comprising a step of performing a quantitative detection of conjugated bilirubin as a biomarker with a diagnostic device;
preferably, the conjugated bilirubin comprises bilirubin β-monoglucuronide and/or bilirubin γ-diglucuronide.

2. The method of claim 1, wherein, the diagnostic device is selected from a medical device, a kit, a test strip and a detection device; wherein the biological sample from a test subject is detected with the diagnostic device;
preferably, the test subject is a newborn; and/or, the biological sample is a blood sample;
more preferably, the blood sample is selected from whole blood, plasma, serum and a dried blood spot, such as a dried blood spot.

3. The method of claim 2, wherein, other detection indicator of the test subject can be optionally combined to assess the risk of neonatal biliary atresia with the diagnostic device, and the other detection indicator is selected from one or more of free bilirubin, biliverdin, γ-glutamyl transferase, MMP-7 and other indicators capable of directly or indirectly diagnosing neonatal biliary atresia.

4. The method of claim 2, wherein, the biological sample is contained in an organic solvent dispersion system;
preferably, the organic solvent of the organic solvent dispersion system is selected from one or more of methanol, ethanol, acetone, propylene glycol, acetonitrile, and a combination thereof; and/or, the supernatant is collected for detection after the biological sample is centrifuged;
and/or, the method for quantitative detection is liquid chromatography tandem mass spectrometry.

5. The method of claim 4, wherein, the organic solvent dispersion system comprises a first stabilizer and a second stabilizer; the weight ratio of the content of the first stabilizer and the second stabilizer in the organic solvent dispersion system is in the range of 1:(1 to 50);
preferably, the weight ratio of the content of the first stabilizer and the second stabilizer in the organic solvent dispersion system is in the range of 1:(10 to 45); and/or, the first stabilizer is selected from one or more of butylated hydroxyanisole (BHA), butylated hydroxytoluene (BHT), propyl gallate (PG), tertiary butylhydroquinone (TBHQ), and a combination thereof; the second stabilizer is ascorbic acid;
more preferably, the weight ratio of the content of the first stabilizer and the second stabilizer in the organic solvent dispersion system is in the range of 1:(20 to 40); and/or, the first stabilizer is butylated hydroxytoluene (BHT) and the second stabilizer is ascorbic acid;
further more preferably, the weight ratio of the content of the first stabilizer and the second stabilizer in the organic solvent dispersion system is in the range of 1:(30 to 40).

6. A method for detection of the content of bilirubin, wherein, the method for detection is a quantitative detection method of liquid chromatography tandem mass spectrometry, the content of bilirubin is the content or total content of any one or more selected from free bilirubin, biliverdin, bilirubin β-monoglucuronide and bilirubin γ-diglucuronide.

7. The method of claim 1, wherein, in the liquid chromatography tandem mass spectrometry, the liquid chromatography is selected from high performance liquid chromatography, ultra high performance liquid chromatography and nanoliter liquid chromatography;
preferably, the liquid chromatography is high performance liquid chromatography, the column of the liquid chromatography is selected from C8 and C18 silica gel-packed columns; and/or, the composition of the mobile phase of the liquid chromatography is: aqueous phase A, selected from ultrapure water with a pH of 3-4.5; organic phase B, selected from one or more of acetonitrile, ethanol, methanol, propylene glycol and isopropanol; and/or, the mass spectrometry is selected from quadrupole mass spectrometry, time-of-flight mass spectrometry, ion hydrazine mass spectrometry and high-resolution orbital hydrazine mass spectrometry;
more preferably, the pH is adjusted by a pH adjuster, the pH adjuster is preferably selected from ammonium acetate-acetic acid, ammonium formate-formic acid, trifluoroacetic acid, and trichloroacetic acid buffer systems; more preferably the ammonium acetate-acetic acid buffer system; and/or, the organic phase B is selected from one or more of acetonitrile, methanol, and isopropanol; the organic phase B is preferably consisted of acetonitrile, methanol, and isopropanol; and/or, the condition and the mode settled for qualitative and quantitative detection of mass spectrometry comprise: selecting electrospray ion source (ESI) and selecting ion scan mode based on the response of a target compound to be detected; selecting a multiple reaction monitoring method (MRM) and setting parameters for the multiple reaction monitoring mode.

8. A stable quantitative detection reagent for bilirubin, wherein, the quantitative detection reagent for bilirubin comprises a bilirubin standard, the bilirubin standard is dispensed in a system containing a first stabilizer and a second stabilizer; the bilirubin standard comprises a standard of any one or more selected from free bilirubin, biliverdin, bilirubin β-monoglucuronide and bilirubin γ-diglucuronide;
preferably, the first stabilizer and the second stabilizer are the first stabilizer and the second stabilizer of claim 5;
more preferably, the system containing the first stabilizer and the second stabilizer is an organic solvent dispersion system containing butylated hydroxytoluene (BHT) and ascorbic acid;
further more preferably, the organic solvent of the organic solvent dispersion system is selected from one or more of methanol, ethanol, acetone, propylene glycol, acetonitrile, and a combination thereof.

9. The stable quantitative detection reagent for bilirubin of claim 8, wherein, the system containing a first stabilizer and a second stabilizer is a solid system, the solid system comprises a solid carrier;
preferably, the solid carrier is a filter paper sheet;
more preferably, the filter paper sheet is selected from analytical filter paper, qualitative analytical filter paper and slow quantitative ashless filter paper; and/or, when the filter paper sheet is used as a solid carrier, the filter paper sheet is pretreated as follows: the filter paper sheet is soaked in an organic solvent dispersion system containing a first stabilizer and a second stabilizer and then dried in the shade.

10. A method for detecting the content of bilirubin or assessing the risk of biliary atresia, infant hepatitis syndrome, α1 antitrypsin deficiency disease or Alagille syndrome in a subject, comprising a step of detecting the content of bilirubin with the quantitative detection reagent of claim 8.

11. A kit for detecting the content of bilirubin or assessing the risk of neonatal biliary atresia, wherein, the kit comprises the quantitative detection reagent for bilirubin of claim 8;
wherein, the content of bilirubin is detected by liquid chromatography tandem mass spectrometry, the content of bilirubin is the content or total content of any one or more selected from free bilirubin, biliverdin, bilirubin β-monoglucuronide and bilirubin γ-diglucuronide;
preferably, the kit comprises the standard and internal standard of any one or more s of free bilirubin, biliverdin, bilirubin β-monoglucuronide and bilirubin γ-diglucuronide;
more preferably, the internal standard comprises a taurine bilirubin standard.

12. The kit of claim 11, wherein, the standard of any one or more of free bilirubin, biliverdin, bilirubin β-monoglucuronide and bilirubin γ-diglucuronide are dispensed in a system containing a first stabilizer and a second stabilizer, respectively;
preferably, the system is selected from a liquid dispersion system containing the standard and a test strip carrying the standard; and/or, the weight ratio of the content of the first stabilizer and the second stabilizer in the organic solvent dispersion system is in the range of 1:(1 to 50);
more preferably, the weight ratio of the content of the first stabilizer and the second stabilizer in the organic solvent dispersion system is in the range of 1:(10 to 45); and/or, the first stabilizer is selected from one or more of butylated hydroxyanisole (BHA), butylated hydroxytoluene (BHT), propyl gallate (PG), tertiary butylhydroquinone (TBHQ), and a combination thereof; the second stabilizer is ascorbic acid;
further more preferably, the weight ratio of the content of the first stabilizer and the second stabilizer in the organic solvent dispersion system is in the range of 1:(20 to 40); and/or, the first stabilizer is butylated hydroxytoluene (BHT) and the second stabilizer is ascorbic acid;
even further more preferably, the weight ratio of the content of the first stabilizer and the second stabilizer in the organic solvent dispersion system is in the range of 1:(30 to 40).

13. A method for assessing the risk of biliary atresia, infantile hepatitis syndrome, α1 antitrypsin deficiency disease and Alagille syndrome of a subject, comprising a step of detecting the content of bilirubin with the kit of claim 11;
preferably, the method further comprises an instruction of the kit, the instruction is defined by a method for detection of the content of bilirubin, wherein, the method for detection is a quantitative detection method of liquid chromatography tandem mass spectrometry, the content of bilirubin is the content or total content of any one or more selected from free bilirubin, biliverdin, bilirubin β-monoglucuronide and bilirubin γ-diglucuronide;
more preferably, in the liquid chromatography tandem mass spectrometry, the liquid chromatography is selected from high performance liquid chromatography, ultra high performance liquid chromatography and nanoliter liquid chromatography;
even more preferably, the liquid chromatography is high performance liquid chromatography, the column of the liquid chromatography is selected from C8 and C18 silica gel-packed columns; and/or, the composition of the mobile phase of the liquid chromatography is: aqueous phase A, selected from ultrapure water with a pH of 3-4.5; organic phase B, selected from one or more of acetonitrile, ethanol, methanol, propylene glycol and isopropanol; and/or, the mass spectrometry is selected from quadrupole mass spectrometry, time-of-flight mass spectrometry, ion hydrazine mass spectrometry and high-resolution orbital hydrazine mass spectrometry;
even further more preferably, the pH is adjusted by a pH adjuster, the pH adjuster is preferably selected from ammonium acetate-acetic acid, ammonium formate-formic acid, trifluoroacetic acid, and trichloroacetic acid buffer systems; more preferably the ammonium acetate-acetic acid buffer system; and/or, the organic phase B is selected from one or more of acetonitrile, methanol, and isopropanol; the organic phase B is preferably consisted of acetonitrile, methanol, and isopropanol; and/or, the condition and the mode settled for qualitative and quantitative detection of mass spectrometry comprise: selecting electrospray ion source (ESI) and selecting ion scan mode based on the response of a target compound to be detected; selecting a multiple reaction monitoring method (MRM) and setting parameters for the multiple reaction monitoring mode.

14. A method for assessing the risk of neonatal biliary atresia, wherein, the kit of claim 11 is used as a kit for assessing the risk of neonatal biliary atresia, comprising:
(1) taking a newborn as a test subject to obtain a biological sample as a test sample;
wherein the biological sample is a blood sample; preferably, the blood sample is selected from whole blood, plasma, serum and a dried blood spot, such as a dried blood spot;
(2) quantifying the expression level of a biomarker in the test sample with a quantitative detection method of liquid chromatography tandem mass spectrometry, the content of bilirubin is the content or total content of any one or more selected from free bilirubin, biliverdin, bilirubin β-monoglucuronide and bilirubin γ-diglucuronide; the biomarker comprises bilirubin β-monoglucuronide and/or bilirubin γ-diglucuronide, and optionally in combination of other detection indicator of the test subject to assess the risk of neonatal biliary atresia, the other detection indicator is selected from free bilirubin, biliverdin, γ-glutamyl transferase, MMP-7 and one or more other indicators capable of directly or indirectly diagnosing neonatal biliary atresia;
(3) analyzing the expression level of the biomarker for risk assessment, wherein the analysis can be a comparison with the cutoff value of a quantitative detection, and the conclusion of the risk assessment can be used in assessing the risk of biliary atresia of the test subject is high or low;
(4) dividing the test subject into a high risk group and a low risk group based on the assessment conclusion, wherein the high risk group indicates the need for further clinical diagnosis to determine whether the test subject has biliary atresia;
preferably, the cutoff value of the quantitative detection in step (3) is a value determined by statistical analysis with the quantitative detection in step (2), wherein the expression level of the biomarker in the test sample of a healthy newborn and of the corresponding biomarker in the test sample of a newborn with biliary atresia is determined in advance.

15. A device for predicting the risk of neonatal biliary atresia by the expression level of a conjugated bilirubin, comprising:
(1) a module for receiving a test sample of a test subject;
(2) a module for detecting data on the expression level of a biomarker; wherein the biomarker comprises at least a conjugated bilirubin, wherein the conjugated bilirubin is selected from bilirubin β-monoglucuronide and/or bilirubin γ-diglucuronide, and optionally comprises free bilirubin, biliverdin, γ-glutamyl transferase, MMP-7 and other clinical indicator capable of diagnosing biliary atresia in the test sample;
(3) a module for generating a risk score based on inputting the expression level of a biomarker to a database, wherein the database comprises a control expression profile associated with the test sample and the method for detection; the control expression profile is derived in advance on the basis of the test sample and the method for detection, which may be expressed as a cutoff value for the biomarker detected; the risk assessment is performed by comparing the expression level of the biomarker in the test sample with the cutoff value of the high performance liquid chromatography tandem mass spectrometry as the method for detection, and the test subject is considered to be at high risk of biliary atresia when the expression level of the biomarker is higher than the cutoff value.

16. The method of claim 6, wherein, in the liquid chromatography tandem mass spectrometry, the liquid chromatography is selected from high performance liquid chromatography, ultra high performance liquid chromatography and nanoliter liquid chromatography;
preferably, the liquid chromatography is high performance liquid chromatography, the column of the liquid chromatography is selected from C8 and C18 silica gel-packed columns; and/or, the composition of the mobile phase of the liquid chromatography is: aqueous phase A, selected from ultrapure water with a pH of 3-4.5; organic phase B, selected from one or more of acetonitrile, ethanol, methanol, propylene glycol and isopropanol; and/or, the mass spectrometry is selected from quadrupole mass spectrometry, time-of-flight mass spectrometry, ion hydrazine mass spectrometry and high-resolution orbital hydrazine mass spectrometry;
more preferably, the pH is adjusted by a pH adjuster, the pH adjuster is preferably selected from ammonium acetate-acetic acid, ammonium formate-formic acid, trifluoroacetic acid, and trichloroacetic acid buffer systems; more preferably the ammonium acetate-acetic acid buffer system; and/or, the organic phase B is selected from one or more of acetonitrile, methanol, and isopropanol; the organic phase B is preferably consisted of acetonitrile, methanol, and isopropanol; and/or, the condition and the mode settled for qualitative and quantitative detection of mass spectrometry comprise: selecting electrospray ion source (ESI) and selecting ion scan mode based on the response of a target compound to be detected; selecting a multiple reaction monitoring method (MRM) and setting parameters for the multiple reaction monitoring mode.
